(19) United States
(12) Patent Application Publication
(10) Pub. No.: US 2023/0287395 A1
(43) Pub. Date: Sep. 14, 2023

(54) MODULAR MICROFLUIDIC DEVICES, SYSTEMS AND METHODS FOR TOTAL RNA ANALYSES

(71) Applicants: CAMBRIDGE ENTERPRISE LIMITED, Cambridge, Cambridgeshire (GB); KONINKLIJKE NEDERLANDSE AKADEMIE VAN WETENSCHAPPEN, Amsterdam (NL)

(72) Inventors: Florian Hollfelder, Campridge Cambridgeshire (GB); Tomasz Kaminski, Campridge Cambridgeshire (GB); Joachim De Jonghe, Campridge Cambridgeshire (GB); Fredrik Salmen, Amsterdam (NL); Alexander Van Oudenaarden, Amsterdam (NL)

(21) Appl. No.: 18/020,391

(22) PCT Filed: Aug. 13, 2021
(86) PCT No.: PCT/GB2021/052112

§ 371 Date: Feb. 8, 2023

(30) Foreign Application Priority Data
Aug. 14, 2020 (GB) 2012730.4

(51) Int. Cl. C12N 15/10; B01L 3/00

(52) U.S. Cl. CPC C12N 15/1065; B01L 3/502761; C12N 15/1096; B01L 2200/0668; B01L 2200/16; B01L 2400/0487

(57) ABSTRACT

Modular microfluidic devices, systems and methods for total RNA analyses The invention relates to microfluidic methods of preparing a sequencing library for analyses of total RNA. The invention also relates to modular microfluidic systems for carrying out these methods.

### FIELD OF THE INVENTION

The present invention relates to microfluidic methods of preparing a sequencing library for analyses of total RNA. The invention also relates to modular microfluidic systems for carrying out these methods.

### BACKGROUND

Current droplet microfluidic methods to analyse RNA from single cells analyse only messenger RNA (mRNA) and some long non-coding RNAs. However, there are many other types of RNA in a cell which contribute to cell function.

Current droplet microfluidic methods also only analyse the ends of the RNA. It is important to know the full length of the RNA to understand alternative splicing, for gene mutation scanning and for precise RNA velocity determination.

### SUMMARY OF THE INVENTION

The inventors have devised microfluidic methods and devices for analysing full length total RNA from a single cell.

In a first aspect, there is provided a method of preparing a sequencing library, the method comprising:
a) encapsulating in a microfluidic droplet:
   a cell or cell structure comprising RNA; and
   lysis and optionally RNA fragmentation reagent;
b) incubating the droplet to release the RNA from the cell or cell structure;
c) optionally fragmenting the RNA in the droplet;
d) adding an RNA tagging reagent into the droplet, wherein the RNA tagging reagent adds an oligonucleotide tag to the RNA;
e) incubating the droplet to allow the RNA to be tagged with the oligonucleotide;
f) hybridizing the oligonucleotide tag of the RNA to a primer adapted to initiate cDNA synthesis (cDNA synthesis primer); and
g) performing reverse transcription to obtain a cDNA sequencing library wherein the cDNA in the cDNA sequencing library comprise a barcode and optionally a UMI.

In a second aspect there is provided a modular microfluidic system for preparing a sequencing library, the modular system comprising:
a) a droplet generation module adapted for encapsulation of cells or cell structures, lysis reagent and optionally beads in microfluidic droplets, the droplet generation module comprising a droplet generation junction in fluid communication with one or more input channels, the one or more input channels for flowing cells, lysis reagent, partitioning fluid and optionally beads into the droplet generation junction
b) a picoinjection module adapted to receive the droplet from the first device, the picoinjection module comprising:
   i) a supply channel, into which microfluidic droplets comprising cell lysate and fragmented RNA can be injected wherein the supply channel comprises a droplet spacer; and
   ii) a picoinjector for injecting RNA tagging reagent into the droplets wherein the picoinjector is in fluid communication with the supply channel and is downstream of the droplet spacer.

In a third aspect, there is provided a method for preparing a sequencing library, the method comprising:
a) encapsulating in a microfluidic droplet:
   i) a cell or cell structure comprising RNA; and
   ii) lysis and RNA tagging reagents, wherein the RNA tagging reagent adds an oligonucleotide tag to the RNA;
b) incubating the droplet to release the RNA from the cell or cell structure and to allow the RNA to be tagged with the oligonucleotide;
c) hybridizing the oligonucleotide tag of the RNA to a reverse transcriptase primer; and
d) performing reverse transcription to obtain a cDNA sequencing library wherein each cDNA in the cDNA sequencing library comprises a UMI and barcode.

### DETAILED DESCRIPTION

References are made to the figures in the following description. However, these references are to help explain the different features of the invention and are not intended to limit these features to the specific embodiments in the figures.

Features of the Method

Ranges

Any of the concentration or volume points given below may be made into ranges to provide a preferred range of concentrations. Percentages are given as percentage volumes, i.e. reagent to droplet, v/v.

Cell

By cell is meant an intact cell.

The cell may be from a eukaryotic or prokaryotic organism.

By cell structure is meant a nuclei or any other organelle which comprises RNA.

As an alternative to a cell, a virion or virus capsule (including bacteriophage) may also be analysed using the methods and systems of the invention. Therefore, “cell or cell structure” throughout the claims may be substituted for virus capsule, virion or bacteriophage.

RNA

By RNA is meant to include
Messenger RNA (mRNA), which is involved in protein coding and is the main information on cell state.

RNA may also additionally include any one or more of the following:
Long non-coding RNA (lncRNA), which is involved in gene regulation and expression.
Micro RNA (miRNA) which is involved in regulation of translation by RNA silencing.
Small nuclear RNA (snRNA) which is involved in splicing.
Small nucleolar RNA (snoRNA) which is involved in guide modifications of RNA
Small Cajal RNA (scaRNA) a form of snoRNA found in Cajal bodies that are involved in the modification of spliceosomal RNA
transfer RNAs (tRNA) involved in translation
tRNA-derived small RNA (tsRNA)
ribozymes, catalytic RNAs found in the ribosome
mitochondrial RNA (Mt RNA), RNA found in the mitochondria
ribosomal RNA (rRNA)
pseudogenes
circular RNA (circRNA)
Enhancer RNA

Sequencing Library

A sequencing library is a pool of nucleic acids, for example cDNA or DNA, which have a barcode and optionally a UMI (Unique Molecular Identifier, which in terms of fragmented nucleic acid can also be known as a unique fragment identifier or UFO allowing them to be sequenced using Next Generation Sequencing methods.

A barcode is a nucleic acid tag added to a set of nucleic acids to identify them as a group. For example, a barcode may be added to the RNA within a single cell to identify the RNA as from that cell.

A UMI is a nucleic acid tag which identifies one particular nucleic acid molecule. That is, the UMIs are different on each barcoded molecule. Incorporating a UMI allows the averaging out the sequencing to account for cDNA molecules which are unevenly amplified providing an accurate quantification of gene expression and reduction in the signal to noise.

The barcode and optionally UMI may be incorporated into the cDNA synthesis primer. Alternatively, the barcode and optionally UMI can be introduced into the cDNA during reverse transcription by incorporating them into a template switching oligonucleotide. A further alternative is to add the barcode and optional UMI by ligation: by 5′ or 3′ RNA ligation; or 5′ or 3′ cDNA ligation.

Microfluidic Droplet

By microfluidic droplet is meant a discrete volume of a first liquid in an immiscible second liquid.

Bead

A bead is an efficient way to incorporate barcodes in a droplet where the bead is a support with barcodes attached. The bead may be any shape. The bead may be a non-dissolvable bead or a dissolvable bead.

Where the bead is non-dissolvable, the barcodes may be attached to the bead via a linker which is cleavable to remove the barcode from the bead. The linker may be cleavable with UV. The barcodes may be removed from the bead at any point in the method by cleaving this linker, for example the barcodes may be removed from the bead after lysis of the cell; after fragmentation; after RNA tagging; or after reverse transcription.

Where the bead is dissolvable, the microfluidic droplet is incubated at the recommended temperature and other conditions required to dissolve the bead at any of the above points in the method as for the non-dissolvable bead.

The bead may be 10-100 μm in diameter, for example, 10, 20, 30, 40, 50, 60, 70, 80, 90 or 100 μm. For example, the bead may be approximately 60-65 μm in diameter.

Measuring the Volume of the Droplet

The amount to be added of each reagent is given below in two ways:
1) The amount to be added using the concentrated mixes of the reagents (i.e. the reagents in the reservoirs coupled to the channels); and
2) The final concentration in the droplet after addition of the concentrated mix. The final concentration allows the amount to be added to be calculated accurately, which varies according to the size of the droplet which is in turn determined by if a bead is present and the size of the cell.

The volume to add to the droplet to achieve the final concentration may be calculated by measuring the volume of the droplet and adding a concentrated mix of the reagent to achieve the final concentration.

The volume of the droplet can be measured using either of the following methods:
1) Measure radius or diameter on imaging system (like a microscope) and then use formula V=4/3πR³ to convert to volume.
2) Use the flow rates and calculate droplet generation frequency using an ultrafast camera and divide the cumulative flow rates by the frequency to get the volume per droplet. Method 2 is more precise.

Frequency of picoinjection and flow rates can be calculated manually or using appropriate software.

Concentrated Reagents

The initial concentrated reagents (i.e. those added to the droplet as a concentrated reagent to be diluted in the droplet) are provided:

TABLE 1
Concentrated mixes for addition to droplets

| Reagent | Concentration of concentrated reagent |
|---|---|
| Lysis and fragmentation | 1.5-30 U/ml of protease |
| | 1-60 mM divalent metal ion (optional) |
| | 0.15-1.5% non-ionic detergent |
| | Preferred: |
| | 4-8 U/ml of protease |
| | 10-20 mM divalent metal ion (optional) |
| | 0.35-0.7% non-ionic detergent |
| RNA repair and | 0.5-9 kU/ml of repair enzyme |
| polyadenylation | 75-800 U/ml of polyadenylation enzyme |
| | 0.003-5 mM ATP |
| | Optionally, DTT 5-25 mM |
| | Preferred: |
| | 2-4 kU/ml of repair enzyme |
| | 160-330 U/ml of polyadenylation enzyme |
| | 0.13 mM-0.25 mM of ATP. |
| | Optionally DTT 15-20 mM |
| Reverse transcriptase | 1-50 KU/ml of reverse transcriptase |
| | 0.02 mM-4 mM dNTPs |
| | Preferred: |
| | 10-30 KU/ml of reverse transcriptase |
| | 0.4 mM-1 mM dNTPs |

For the concentrated lysis and fragmentation reagent, the protease may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 U/ml. For the divalent metal ion, the concentration may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mM.

For the non-ionic detergent, the concentration in the concentrated reagent may be: 0.15, 0.2, 0.25, 0.3, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45 or 1.5% v/v.

In cases where the RNA is already fragmented and no further fragmentation is required (e.g. when a sample has been stored at a temperature that causes partial RNA degradation), the divalent metal ion (normally responsible for fragmentation) may be removed from the reagent list. The concentrations of the other reagents remain the same. The volume to be added to the cell suspension remains the same also.

For the RNA repair and polyadenylation reagent, the concentrated reagent concentrations may be:
1.5, 2, 2.5, 3, 3.5, 4 or 4.5 kU/ml of repair enzyme.
75, 100, 125, 150, 175, 200, 225, 250, 275, 300, 325, 350 or 375 U/ml of polyadenylation enzyme.
0.003, 0.01, 0.05, 0.1, 0.15, 0.2, 0.25, 0.3, 0.35, 0.4, 0.5, 0.6, 0.7, 0.8, 0.9, 1, 2 or 3 mM ATP.

For the reverse transcriptase, the concentrated reagent may be: 10, 15, 20, 25, 30, 35 or 40 kU/ml.

Therefore, these concentrated reagents may be used in the amounts specified in the claims and set out in detail below.

The amount of reagents added depends on the workflow, i.e. if a bead is added during encapsulation which forms a larger droplet requiring a larger amount of reagent to be added; or if the bead is added later in the workflow (the concentrations in the droplet are the same for both workflows shown in the figures, and these “in droplet” concentrations are provided below).

TABLE 2
Amount of concentrated reagent added

| Concentrated | Bead added during | Bead added during droplet |
|---|---|---|
| reagent | encapsulation (FIG. 1) | fusion (FIG. 5) |
| Lysis and | 0.05-0.4 nl, for | 5-25 pl, for example |
| fragmentation | example, 0.05, 0.1, | 5, 6, 7, 8, 9, 10, 11, 12, 13, |
| reagent | 0.2, 0.3 or 0.4 nl | 14, 15, 16, 17, 18, 19, 20, |
| | | 21, 22, 23, 24, 25 pl |
| Repair and | 0.1-0.5 nl, for | 5-50 pl, for example, 5, 6, 7, |
| polyadenylation | example, 0.1, 0.2, | 8, 9, 10, 11, 12, 13, 14, 15, |
| reagent | 0.3, 0.4 or 0.5 nl | 16, 17, 18, 19, 20, 21, 22, 23, |
| | | 24, 25, 26, 27, 28, 29, 30, 31, |
| | | 32, 33, 34, 35, 36, 37, 38, 39, |
| | | 40, 41, 42, 43, 44, 45, 46, 47, |
| | | 48, 49 or 50 pl |
| Reverse | 0.1-1.5 nl, for | 0.1-1.5 nl, for example, 0.1, |
| transcriptase | example, 0.5, 0.6, | 0.2, 0.3, 0.4, 0.5, 0.6, 0.7, |
| reagent | 0.7, 0.8, 0.9, 1, | 0.8, 0.9, 1, 1.1, 1.2, 1.3, 1.4 |
| | 1.1, 1.2, 1.3, 1.4 | or 1.5 nl |
| | or 1.5 nl | |

TABLE 3
The ratio of volume of the concentrated
reagent added to the size of the droplet.

| Concentrated | Bead added during | Bead added during droplet |
|---|---|---|
| reagent | encapsulation (FIG. 1) | fusion (FIG. 5) |
| Lysis and | 20-50% the volume of | 20-75% the volume of |
| fragmentation | the droplet. For example, | the droplet. For example, |
| reagent | 20, 30, 40 or 50% | 20, 30, 40, 50, 60, 70 or |
| | | 75% |
| Repair and | 10-50% the volume of | 10-50% the volume of |
| polyadenylation | the droplet. For example, | the droplet. For example, |
| reagent | 10, 20, 30, 40 or 50% | 10, 20, 30, 40 or 50% |
| Reverse | 25-150% the volume of | 150-500% the volume of |
| transcriptase | the droplet. For example, | the droplet. For example, |
| reagent | 25, 30, 40, 50, 60, 70, | 150, 200, 250, 300, 350, |
| | 80, 90, 100, 110, 120, | 400, 450 or 500% |
| | 130, 140 or 150% | |

The amount or volume ratio for the lysis and fragmentation reagent refers to that in the final droplet as the droplet is formed during the encapsulation step which encompasses this reagent. The amount or volume ratio can be input into the droplet by adjusting the flow rates of the different reagents into the encapsulation channel to form the droplet.

The amount or volume ratio for the repair and polyadenylation reagent and reverse transcriptase reagent is the amount added to the incoming droplet or the volume ratio compared to the incoming droplet.

Guide sizes of droplets in microns and by volume are given below. This is based on human HEK293T cells.

TABLE 4
size in microns

| | VASA-drop | superVasa |
|---|---|---|
| | (Radius of | (Radius of |
| | droplet in μm) | droplet in μm) |
| After encapsulation | 52.3 | 19.3 |
| After pico-injection of repair | 57.6 | 21.3 |
| and poly(A) tailing mix | | |
| After pico-injection of | 72.6 | 62.9 |
| RT/merging with RT droplet | | |

TABLE 5
size in volumes

| | VASA-drop | superVasa |
|---|---|---|
| | (Volume) (bead | (Volume) (bead added |
| | added during | by droplet fusion in |
| | encapsulation) | the third module) |
| After encapsulation | 0.6 nl | 30 pl |
| After pico-injection of repair | 0.8 nl | 40 pl |
| and poly(A) tailing mix | | |
| After pico-injection of | 1.6 nl | ~1 nl |
| RT/merging with RT droplet | | |

Diluted Reagents in the Droplet

The concentrations referred to below refer to “in droplet concentrations”, i.e. AFTER the concentrated reagents have been diluted in the droplet. A table summarising these concentrations in the droplet is provided below.

TABLE 6
Concentration in the droplet after each microfluidic step

| | Concentration in droplet after addition |
|---|---|
| Reagent | of each reagent |
| Lysis and fragmentation | 0.2-10 U/ml of protease |
| | 0.5-20 mM divalent metal ion (optional in |
| | cases where the RNA is already fragmented, |
| | e.g. due to sample storage conditions) |
| | 0.05-1% non-ionic detergent |
| | Preferred: |
| | 0.5-5 U/ml of protease |
| | 4-12 mM divalent metal ion (optional) |
| | 0.1-0.5% non-ionic detergent |
| RNA repair and | 100 U/ml-4 kU/ml of repair enzyme |
| polyadenylation | 10-500 U/ml of polyadenylation enzyme |
| | 0.001-5 mM ATP |
| | Optionally DTT 0.5-25 mM |
| | Preferred: |
| | 500 U/ml-1 kU/ml of repair enzyme |
| | 50-400 U/ml of polyadenylation enzyme |
| | 0.02 mM-0.1 mM of ATP. |
| | Optionally DTT 1-10 mM |
| Reverse transcriptase | 1-40 kU/ml of reverse transcriptase |
| | 0.01 mM-2 mM dNTPs |
| | Preferred: 5-25 KU/ml |
| | 0.2 mM-0.7 mM dNTPs |

Lysis and Fragmentation Reagent

The purpose of the lysis reagent is to lyse the cell or cell structure to release the RNA. The purpose of the fragmentation reagent is to fragment the RNA.

The lysis and fragmentation reagent may comprise any one or more of the following:
a) a protease;
b) a divalent metal ion;
c) a non-ionic detergent;

optionally wherein the lysis and fragmentation reagent is added to the droplet to result in any one or more of the following concentrations in the droplet:
a) 0.5-30 U/ml of protease;
b) 0.5-40 mM of divalent metal ion; and/or
c) 0.05-1.5% v/v of non-ionic detergent.

For example, the protease may be added to a concentration of 1, 2, 3, 4, 5, 6, 7, 8, 9, or 10 U/ml. Preferably, the protease is added to a concentration of 0.5-5 U/ml.

The protease may be Proteinase K.

The lysis agent may alternatively comprise lysozyme instead of a protease if the cell being analysed is a bacterial cell.

The divalent metal ion may be Mg²⁺, Mn²⁺, Ca²⁺ or Zn²⁺. Preferably the divalent cation is Mg²⁺. The concentration of the divalent metal ion may be 1, 2, 3, 4, 5, 6, 7, 8, 9, 10, 11, 12, 13, 14, 15, 16, 17, 18, 19 or 20 mM. Preferably the ion is added to a concentration of 4-12 mM.

The non-ionic detergent may be any which is compatible with the downstream reactions. For example, Triton X-100 or IGEPAL-CA630. The concentration may be 0.05, 0.1, 0.15. 0.2, 0.25, 0.3, 0.35, 0.4, 0.45, 0.5, 0.55, 0.6, 0.65, 0.7, 0.75, 0.8, 0.85, 0.9, 0.95 or 1%. Preferably, the detergent concentration is added to a concentration of 0.1-0.5% in the droplet.

The cell suspension may additionally comprise a density gradient medium, for example Optiprep™, which prevents cell sedimentation. The concentration of the density gradient medium in the droplet after encapsulation may be 1-15%, for example, 1, 2, 3, 4, 5, 6, 7, 8, 9, 10, 11, 12, 13, 14 or 15%, optionally approximately 3-8% for whole cells and 1-4% for nuclei.

The lysis reagent may additionally comprise any of the following: a buffering agent for enzyme stability, e.g. Tris HCl at approximately pH 8 and/or PBS, salt, e.g. KCl, dNTPs and Tween.

RNA Tagging Reagent

The purpose of the RNA tagging reagent is to add a tag to the RNA. Repair of the RNA may also be carried out by the RNA tagging reagent. By tag is meant a tag which is covalently bound to the RNA. Therefore, tagging covalently binds a tag, for example an oligonucleotide, to the RNA.

The RNA may be tagged by any known method in the art. For example, by ligation of an oligonucleotide tag (which hybridizes with the cDNA synthesis primer to be used in the subsequent reverse transcription step), for example to the 3′ end of the RNA by an RNA ligase, for example T4 RNA ligase.

The oligonucleotide tag may be of any sequence and length. For example, 6-25 bp. The sequence of the tag is chosen to have none or very little secondary structure, and have a melting temperature in the working temperature range of the reverse transcriptase. The tag also should not form primer dimers or hybridize to any barcodes or adaptors used in downstream sequencing; and also does not bind to any sequence being analysed.

For example, a poly-G or poly-U tag may be added.

The RNA tagging reagent may be an RNA repair and polyadenylation reagent.

RNA Repair and Polyadenylation Reagent

The RNA repair and polyadenylation reagent comprises the essential enzymes and substrates required to end repair the fragmented RNA, i.e. to add an OH group at the 3′ end; and polyadenylate the RNA, i.e. add a plurality of adenines to the 3′ end.

The RNA repair enzyme may be T4 Polynucleotide Kinase (T4 PNK) or any other enzyme capable of adding an OH group at the 3′ end of the RNA, and compatible with the polyadenylation enzyme.

The polyadenylation may be performed by any enzyme capable of adding a poly(A) tail to the 3′ end of RNA. For example, the enzyme may be poly(A) polymerase or poly(U) polymerase (a poly(U) polymerase can also be used to add a poly-U or poly-G tag; yeast or other poly(A) polymerase may also be used to add a poly-G tag following repair when specifically a poly-A tag is not added and instead a poly-G or poly-U tag is added). The enzymes may be derived from any organism, for example E. coli or yeast poly(A) polymerase.

The RNA repair and polyadenylation reagent may also comprise ATP (alternatively the ATP may be added in the encapsulation reagent). A reducing agent, for example DTT, may also be added.

Concentrations for the reagents in the droplet after addition of the reagent are as follows:
The polyadenylation polymerase may be at a concentration of 10-500 U/ml. For example, 50, 100, 150, 200, 250, 300, 350, 400, 450 or 500 U/ml;
The RNA repair enzyme may be at a concentration of 0.1-4 kU/ml, for example 0.5, 0.6, 0.7, 0.8, 0.9, 1, 1.1, 1.2, 1.3, 1.4 or 1.5 kU/ml;
The ATP may be at a concentration of 0.001-5 mM ATP, for example 0.001, 0.01, 0.02, 0.03, 0.04, 0.05, 0.1, 0.2, 0.3, 0.4, 0.5, 0.6, 0.7, 0.8, 0.9, 1, 1.5, 2, 2.5, 3, 3.5, 4, 4.5 or 5 mM.

Ideally, the RNA repair and polyadenylation reagent in the droplet comprises the following:
Polyadenylation polymerase at a concentration of 50-400 U/ml
RNA repair enzyme at a concentration of 0.5-1 kU/ml
ATP at a concentration of 0.02-0.1 mM

The RNA repair reagent may additionally comprise: Tris-Hcl, DTT, KCl, MgCl2 and an RNA degradation inhibitor, for example RnaseOut™

Reverse Transcriptase Reagent for Performing Reverse Transcription

The reverse transcriptase reagent may comprise 1 or more reverse transcriptases (RT). The reverse transcriptase may comprise template switching activity. The reverse transcriptase may alternatively or additionally function to process long sequences of RNA. Two or more reverse transcriptases may be used to produce a mix with both these functions.

The concentration of the RT in the droplet after adding the RT reagent may be: 1-40 kU/ml. For example, 1, 5, 10, 15, 20, 25, 30, 35 or 40 kU/ml.

The concentration of dNTPs for reverse transcription is: 0.01 mM-2 mM, for example, 0.1, 0.5, 1, 1.5 or 2 mM. This concentration is required for reverse transcription. However, the dNTPs may be added at an earlier step, for example, encapsulation, or polyadenylation.

The reverse transcriptase reagent may additionally comprise any one or more of the following: A divalent metal ion, e.g. MgCl₂, a reducing agent, e.g. DTT, and buffering and salts for stability of the enzymes.

Primer Adapted to Initiate cDNA Synthesis (cDNA Synthesis Primer)

The primer may be any which is designed to bind to the oligonucleotide tag described above. The primer may be 4-60 bp in length, for example, 4, 6, 8, 10, 15, 20, 25, 30, 35, 40, 45, 50, 55 or 60 bp.

When RNA repair and adenylation reagent is used as the RNA tagging reagent, a primer which binds to the polyA tail of the mRNA for example a poly T primer, is used. The poly-T primer comprises all Ts, and is at least 6, 7, 8, 9, 10, 15, 20, 25, 30, 35 or 40 nucleotides.

The cDNA synthesis primer may additionally comprise the barcode and optionally a UMI. The cDNA synthesis primer may be added via a bead.

TSO

A template switching oligonucleotide or TSO may be used to initiate second strand DNA synthesis as efficiently as possible.

The TSO comprises 3 riboGuanosines that can hybridize to the dC tail added by a reverse transcriptase with template switching activity, for example M-MLV-RT. This allows the RT enzyme to further fill-in a PCR adapter at the 3′ of the cDNA molecule which then allows for second strand synthesis using PCR and/or amplification.

In addition to 3 riboguanosines, the TSO comprises a primer sequence which is used as a handle to PCR the cDNA. The primer can be any primer sequence suitable for PCR. For example, if using Illumina NGS, the primer would be a Read 1 adaptor sequence.

The 5′ of the TSO may be blocked, for example with biotin or any other bulky molecule which prevents concatenation. The terminal guanosine may also be a locked-nucleic acid (LNA).

An example TSO is: 5′-biotin-PCR primer— rGrG(+G)-3′ (where rG is riboguanosine and (+)G is the locked guanosine). This is used for PCR amplification of the cDNA (to provide a second handle), therefore the PCR primer section can be any primer sequence suitable for PCR (i.e. no secondary structure, melting temperature compatible with the barcode, no self-priming or primer-dimer formation). A barcode and/or UMI may also be included in the TSO. The LNA blocking also blocks polyadenylation.

Picoinjection

Picoinjection directly dispenses reagents into microfluidic droplets. This is in contrast to droplet fusion (an alternative way of adding reagents into droplets) which electro-coalesces pairs of droplets, one of the droplets containing the substrates for the reaction; the other second droplet containing the reagents for the reaction.

Picoinjection is carried out using a picoinjector. A picoinjector is a channel comprising the reagents to be added to the microfluidic droplet and a pair of electrodes. At the intersection of the picoinjector and the supply channel there are electrodes which induce an electric field that perturbate the surface tension of the droplet via coalescence. This allows for the injected solution to be compartmentalized inside the droplet. The injected solution then merges with the rest of the droplet when it moves away from the electrode, in the direction of the flow.

By adjusting the flow rate the volume of reagent added can be precisely controlled.

Self-triggering picoinjectors can also be employed which detect the microfluidic droplet as it flows across the picoinjector. The detection signal then triggers the electrodes which destabilizes the water/oil interfaces allowing the reagent to enter the droplet.

Frequency

The frequency is the number of droplets picoinjected per second.

This may be 20-10 kHz. For the workflow in FIG. 1, the frequency may be approximately 300 Hz.

For the frequency of the workflow in FIG. 5 may be approximately 2 kHz.

Droplet Fusion

Droplet fusion uses electro-coalescence to merge droplets by applying an electric field to destabilize the droplets-oil interfaces.

Collection Device

To collect the droplets from the droplet generation device, to facilitate incubation and for reinjection, a collection device may be used. The collection device comprises a container, the container comprising an immiscible liquid with lower density than water, optionally a hydrocarbon or silicone oil, the container comprising a tip, wherein the tip is connected to the exit of the droplet generation module and wherein the container is connectable to a pump, wherein the pump optionally aspirates the droplets into the container via the tip;
b) the microfluidic droplets are incubated to lyse the cell; and
c) optionally the droplets are reinjected into the picoinjection device by connecting the container to the pump adapted to eject droplets from the tip.

By using one container to collect, incubate and reinject droplets, this reduces merging of the droplets which can be caused by the droplets travelling along lengths of connective tubing often used for collection. FIG. 1 shows an example of a device. A syringe is filled with an oil compatible with the droplets for re-injection of the droplets back into the second device. The collection device may be of a transparency to allow UV radiation to penetrate into the droplets in the device. This allows cleavage of UV-cleavable linkers which bind the primers to the bead.

Modular Method for RNA Analyses

The following steps are described with reference to FIG. 1 which shows a modular method implemented by the system described below. By modular is meant that certain steps of the reaction are carried out independently of other steps allowing optimisation of each step.

Encapsulating in a Microfluidic Droplet:

A microfluidic droplet is formed which contains: a cell or cell structure; and lysis and fragmentation reagent. A bead can also be included in the droplet as shown in FIG. 1. The bead may comprise the cDNA synthesis primers.

After encapsulation, the droplets are collected (for example using the collection device described above). The droplet containing the cell, lysis and fragmentation reagent and optionally a bead, is then incubated to release the RNA and fragment the RNA.

Incubation of the droplet to release and fragment the RNA: The collected droplet can be incubated at room temperature (16-25° C.) for approximately 25 minutes, for example 25-40 minutes. During this incubation, the RNA is released from the cell/cell structure. This is followed by a second incubation of at least 70° C. for at least 5 minutes (for example 2 minutes up to 2 hours) to fragment the RNA. Longer incubations mean more fragmentation. The incubation may be in a water bath as shown. The incubation to fragment the RNA may not be required, if fragmentation is not required (as explained above).

This may then be followed by an ice bath for at least 5 minutes to stop the fragmentation reaction.

This incubation releases the RNA from the cell or cell structure and fragments the RNA into nucleotides of approximately 100 bp-3000 bp, optionally 100-2000, or 100-1000 or 100-500 bp. Fragmenting the RNA into smaller sizes allows the entire length of the RNA to be sequenced. If the RNA is longer, sequencing methods do not stretch the entire length of the RNA.

FIG. 2 shows the molecular process occurring during the workflow of FIG. 1.

After or during incubation to lyse the cells and fragment the RNA, the cDNA synthesis primers may be released from the bead by UV incubation for example if the cleavable linker to the bead is photo-cleavable as shown in FIG. 1. Alternatively, the cDNA synthesis primers may be cleaved from the bead later in the method, or may not be cleaved, for example where reverse transcription is carried out after de-emulsification (which is described below). The bead may also be a dissolvable bead not requiring cleavage.

Adding an RNA tagging reagent into the droplet: The microfluidic droplet now comprises RNA fragments. These are tagged by adding an oligonucleotide tag to the 3′end or 5′end of the RNA. The oligonucleotide tag is one which hybridizes with the cDNA synthesis primer. For example, a polyA tag may be added to the 3′ end of the RNA following RNA repair as shown in the second step of FIG. 1. The RNA repair enzyme adds an OH group at the 3′ end. The RNA is then ready for polyadenylation which adds a plurality of adenines to the 3′ end of the fragmented RNA.

In FIG. 1, the RNA tagging reagent (polyA mix: the repair and polyadenylation reagent described above) is added by picoinjection.

The microfluidic droplet containing the RNA tagging reagent is then incubated to allow the RNA tagging reaction to proceed.

Incubating the droplet to allow the RNA to be tagged with the oligonucleotide: When tagging by polyadenylation, incubation may be at room temperature, for example 16-25° C. for at least 20 minutes, for example 20-40 minutes, followed by incubation at a 37C for approximately 8 minutes, for example 5-15 minutes. Incubation may be in a water bath. The reaction is stopped by using an optional ice bath for at least 2 minutes.

Hybridizing the oligonucleotide tag of the RNA to a primer adapted to initiate cDNA synthesis: Any RNA which already has a poly-A tail will hybridize after encapsulation with the poly-T primer on the bead. For RNA which does not already have an RNA tail, the RNA will hybridize to the poly-T primer once the poly-A tail has been added to the RNA following RNA repair and polyadenylation. Hybridization may require incubation at room temperature or up to 50° C.

The tagged RNA from the second microfluidic device after incubation, is reinjected into the third microfluidic device. Reverse transcriptase reagent is then added by picoinjection. The droplets are then collected.

Performing reverse transcription to obtain a cDNA library: The droplets are then incubated to allow reverse transcription to proceed. This incubation may be at the following temperatures:
approximately 50° C., for example at least 40° C. for approximately 2 hours, for example at least 1 hour.
Alternatively, if there is more than 1 reverse transcriptase (for example where two or more are chosen for different functions, e.g. template swapping activity) different temperatures for reverse transcription may be used to allow each enzyme to function optimally. For example, as a guide the following were used with a combination of maxima and superscript III: approximately 42° C. for 1 hour; followed by approximately 50° C. for 30 minutes; followed by ten×2-minute cycles; and finally approximately 70° C. for 20 minutes.

A second strand of DNA may then be synthesised for PCR. This is most efficiently carried out by using a TSO as explained above.

Each droplet now contains whole cell RNA from a single cell with a barcode which identifies the RNA as from that cell.

The droplets are then pooled and de-emulsified. This may be done as follows:

The surfactant oil is aspirated and 50 μl of nuclease-free water is added to the tube. Then 200 μl of 1H,1H, 2H,2H-perfluoro-1-octanol is added to the tube and the latter is spun down on a tabletop centrifuge for 30 seconds. The oil phase is then aspirated and discarded, the tube can then be stored at −80° C. until further library preparation.

Further library preparation may involve any one or more of the following steps:
Second strand synthesis;
Adapter ligation
Adapter hybridization
Depletion of DNA oligonucleotides
Depletion of the rRNA or other targeted molecules
cDNA synthesis
PCR or other amplification.

By ligating the adapter first and then depleting the amplified rRNA (aRNA, generated through in vitro transcription amplification of the cDNA libraries), this allows for more effective removal of the undigested short rRNA fragments.

Sorting step wherein the sorting step comprises dividing the droplets into a first droplet and a second droplet:

Any of the modular microfluidic devices or droplet fusion module may have a sorter. The microfluidic sorter is described below. The sorting step divides the droplets into a first droplet set and a second droplet set. The sorting may divide the droplets into those containing cell lysate from live cells (first droplet set) and those containing lysate from dead cells or empty droplets containing no cells (second droplet set). Removing the dead cells and empty cells and cell doublets from the analyses increases the signal to noise ratio allowing for greater depth of sequencing to be achieved and increased confidence in downstream bioinformatic processing of the dataset. Therefore, after the sorting step, the droplets comprising live cells are selected for further processing in the method; the droplets which comprise dead cells and/or droplets with more than 1 cell or cell structure and/or droplets containing no cell or cell structure are discarded. The sorting step may be downstream of encapsulation as shown in FIG. 5. The sorter may also be downstream of picoinjection in the second microfluidic device. Alternatively, the sorting step can be downstream of picoinjection in the third device.

With regards to removing doublets, this can be done as the signal from two cells is summed up and, in most cases, is higher than the signal from a single cell. Also, if cells are not located within a close proximity inside a droplet, then the duration of signal is larger, and those long signals can be also discarded during sorting. Fluorescence readout can be also combined with image analysis to discard cell doublets and multicellular aggregates.

Cells may also be sorted in different types by using antibody binding or using reporter cell lines. This can be done by incubating the cells prior to encapsulation (injection into the first device) with an antibody which binds one a subset of the cells. With regards to cell reporter lines, specific cell types that may harbour intracellular fluorescent proteins or sensors encoding for a desired phenotype can be sorted for enrichment from the pool of cells.

Sorting may require an initial step before encapsulation of the cells in the droplet of staining the cells as shown in FIG. 5.

FIG. 5 shows an alternative workflow where the bead is added during droplet fusion to add the reverse transcriptase reagent.

As described above for FIG. 2, this workflow shows encapsulation and RNA tagging.

A sorter is also shown downstream from encapsulation. The sorter uses staining (therefore any of the methods may comprise a pre-step of staining the cells to be analysed with a stain which identifies live cells from dead cells) of live cells with Calcein-AM stain to sort live cells from dead cells (or empty droplets which also will not be stained). Other methods of sorting can also be used, for example image-based sorting.

The difference with the method of FIG. 1 is that the bead and reverse transcriptase reagent are added via droplet fusion. Therefore, in the first microfluidic device, lysis and fragmentation reagent is added to the cells to form smaller droplets (as the droplets do not contain the bead). The use of smaller droplets (as there is no bead) at the beginning of the method allows higher throughput in these devices.

Therefore, the method comprises encapsulating the bead during droplet fusion when adding the reverse transcriptase reagent. As for the method above, one or more than one reverse transcriptase may be added by droplet fusion.

Features of the Modular Microfluidic System for RNA Analyses

A modular microfluidic system is provided adapted to perform the above workflows. This system will be described with reference to FIG. 1.

The First Microfluidic Device (Droplet Generation Module)

The first microfluidic device or “droplet generation module” will be described by reference to the example illustration in FIG. 1 (top).

The droplet generation microfluidic module can use flow focusing, step emulsification or cross flowing droplet formation to form the droplets. Examples of droplet generation modules using flow focusing and having different channel geometries are shown in FIGS. 1, 5 and 6. These are discussed below only to aid explanation.

The droplet generation module may comprise a droplet generation junction in fluid communication with one or more channels, the channel(s) adapted to flow cells, lysis reagent, a partitioning fluid, e.g. oil, and optionally a bead into the droplet generation junction. The droplet generation junction is adapted to encapsulate a cell, lysis reagent and optionally a bead in the partitioning fluid.

For step emulsification, the droplet generation junction may comprise the droplet generation junction may comprise microchannel or parallel microchannels that enter the deep outer continuous phase reservoir. The phase to be dispersed spontaneously breaks into droplets at a step change in the height of a microchannel.

For cross flowing droplet formation, the droplet generator may comprise a T or Y junction. Most commonly, the channels are perpendicular in a T-shaped junction with the phase to be dispersed (cells, lysis reagent, beads) intersecting the continuous phase (partitioning fluid).

In flow focusing, a partitioning channel is included which is adapted to flow partitioning fluid across the flow of cells, lysis reagent and beads. The partitioning channel may be at an angle, for example approximately perpendicular to the flow of cells, lysis reagent and optionally beads. For flow focusing the junction may additionally comprise a constraint, e.g. a narrowing of the channel which exits the junction which aids formation of a droplet from the cell, lysis reagent and optionally bead phase to be dispersed.

An example of a droplet generation module which uses flow focusing is provided in FIGS. 1 and 5: “first microfluidic device”. The module comprises an encapsulation channel into which cells or cell structures (for example nuclei), lysis reagent and optionally a bead are injected.

The cells and lysis reagent and beads may be injected into the encapsulation channel via different channels. Or the cells may be injected into the encapsulation channel as shown. The beads may also be injected into the encapsulation channel via the lysis channel.

FIG. 1 shows these different channels (encapsulation (into which the cells are injected), lysis reagent channel as well as a bead channel). Downstream of these channels, the module comprises a partitioning channel into which fluid which aids droplet formation, for example oil, is injected. Downstream of this partitioning channel there is a droplet generation junction. The droplet generation junction is found at the intersection between the partitioning oil channels and the encapsulation channel. The droplet form as the aqueous fluid is pushed through the wall of partitioning fluid formed as it flows into the encapsulation channel.

The droplet generation module may comprise:
an encapsulation channel into which cells or cell structures (for example nuclei) are injected;
a lysis and fragmentation reagent channel; and
optionally a bead injecting channel (alternatively the bead can be injected using the lysis reagent channel).

FIG. 1 (top) shows the lysis and cell encapsulation channels. Downstream of these channels, the module comprises a partitioning channel into which fluid which aids droplet formation, for example oil, is injected. Downstream of this partitioning channel there is a droplet generation junction. The droplet generation junction is found between the partitioning oil channels and the supply channel. The droplet form as the aqueous fluid is pushed through the wall of partitioning fluid formed as it flows into the supply channel, typically at an angle into the encapsulation channel (for example perpendicular to the flow in the encapsulation channel). Other methods for forming droplets are also known in the art and may be used in the droplet generation module.

After the droplet is generated, the droplet can be further processed using the picoinjection module (FIG. 6, bottom left).

The droplet generation module may further comprise a sorter, downstream from the droplet generation junction, the sorter comprising a bifurcated sorting junction downstream of the droplet generation junction, the bifurcated sorting junction in fluid communication with a first exit channel and a second exit channel wherein the bifurcated sorting junction is adapted to sort the droplets into a first droplet set which exits via the first exit channel and a second droplet set which exits via the second exit channel. For example, the sorting may divide the droplets into those containing the lysate of live cells (first droplet set) and those containing lysate from more than 1 cell, dead cells or empty droplets containing no cells (second droplet set). The droplets comprising 1 live cell or 1 cell structure, may be collected and further processed.

Using Calcein-AM as shown in FIG. 5 to stain the live cells, the following protocol can be followed: the light from the 488 nm laser was delivered to the sorting junction by the excitation light optical fiber. The emission light emerging from the detection optical fiber connected to the detector tube housing a set of emission filters mounted before the detector of photomultiplier tube. When a fluorescence light signal was higher than an arbitrarily set threshold a high voltage pulse was generated (1 kV) by a set of electronic devices including a high voltage amplifier and delivered to the microfluidic sorting junction by ‘salt electrodes’ filled with 5M NaCl solution. As a result, highly fluorescent droplets with live cells were derailed to the collection channel for positive ‘hits’. The duration and delay of pulse can be modified according to the flow rates and the desired throughput of the sorting.

The first channel further comprises a droplet channel, in fluid communication with the first exit channel and adapted to add empty droplets to the droplets to be analysed to bulk out the sample.

Towards the end of the first and second exit channels of the sorter, the diameter of the channels may increase. This is to prevent merging of the droplets which can occur when moving from the small in diameter exit channels to a wide tubing for example without a gradual increase in the diameter of the channels towards the end of the channels where the droplets are collected.

Diameter is used as a measurement of the distance from one side of the channel to the other side of the channel; the length of the line bisecting the cross-sectional area of the channel. The channels may be tubes which are square, circular or rectangular in cross section. Diameter is used as the measurement for all of these possible geometries. For example, where the channel is square or rectangular in cross-section, the diameter refers to the width and depth of the channel.

The width and/or depth of the sorting junction may be the same diameter or larger than the diameter of the droplet. For example, the width and depth of the sorting junction may be 150-200 μm (for beads which are approximately 60-65 μm in diameter). For example, as a guide, for VASAdrop (where the bead is incorporated into the microfluidic droplet in the first step of the method), the encapsulation channel may have a depth of approximately 80 μm; the detection spot may be where the channel is approximately 90-100 μm deep; and the sorting junction may have a depth of approximately 180 μm. These dimensions are a guide for when the cell is a human cell.

For superVASA, where the bead is incorporated in later steps, the droplets from the encapsulation step are smaller (around 30 pl in volume or 38 μm in diameter). The width and depth of the sorting junction therefore may be approximately 70-120 μm, for example 90-110 μm.

Therefore, the width and depth of the sorting junction may be 1-3 times the diameter of the droplet.

By making the sorting junction deeper, this allows more efficient droplet sorting as the droplets can be pulled by the electric field for example more efficiently meaning higher throughput.

The deeper sorting junction (the sorting junction with the larger width and depth compared to the droplet diameter) can be applied to any of the droplet sorters described.

The mechanism of sorting may be by pre-staining cells to provide sorting by fluorescence. As an alternative to emission, sorting may be by example image analysis, fluorescence anisotropy, absorbance or Raman scattering activated sorting (including SERS—Surface Enhanced Raman Scattering and SRS—Stimulated Raman Scattering).

By system is meant two devices which work in tandem. The first device is a droplet generation module. This provides the microfluidic droplets which are injected into the picoinjection or second device below. The droplets collected from the second device are then collected and injected into the third device. The collected droplets are incubated to allow lysis and RNA tagging between modules as explained in the method above. ‘Module’ refers to a device which can carry out part of a workflow. The modularity may be provided by physically separate devices.

The Second Microfluidic Device (Picoinjection Module)

An example of the second device is shown in FIG. 6 (bottom left). This second module is a picoinjection module adapted to receive the droplet from the first device, the picoinjection module comprising:
i) a supply channel, into which microfluidic droplets comprising cell lysate and fragmented RNA can be injected wherein the supply channel comprises a droplet spacer; and
ii) a picoinjector for injecting RNA tagging reagent into the droplets wherein the picoinjector is in fluid communication with the supply channel and is downstream of the droplet spacer.

The module comprises a supply or inlet channel into which the microfluidic droplet is injected. The supply channel also comprises a droplet spacer. The function of the droplet spacer is to add spacer oil to evenly space out the droplets prior to picoinjection. The droplet spacer may comprise an auxiliary channel in fluid communication with the supply channel wherein in use the auxiliary channel is attached to a reservoir of spacer oil.

Downstream of the droplet spacer (by downstream and upstream throughout the specification refers to when in use, the flow through the device in normal use) there is a picoinjector.

A picoinjector is a channel comprising the reagents to be added to the microfluidic droplet and a pair of electrodes. At the intersection of the picoinjector and the supply channel there are electrodes which induce an electric field that perturbate the surface tension of the droplet via coalescence. This allows for the injected solution to be compartmentalized inside the droplet. The injected solution then merges with the rest of the droplet when it moves away from the electrode, in the direction of the flow. By adjusting the flow rate, the volume of reagent added can be precisely controlled.

Self-triggering picoinjectors can also be employed which detect the microfluidic droplet as it flows across the picoinjector. The detection signal then triggers the electrodes which destabilize the water/oil interfaces allowing the reagent to enter the droplet.

The module may additionally comprise a dilution channel upstream of the spacer. This is shown by example in FIG. 3. The dilution channel is configured to add oil to the droplets upon injection into the device and upstream of the spacer. Diluting oil reduces the packing of the emulsion and prevent shearing of droplets and provide smooth arrangement of droplets in the narrowing chamber before they are evenly spaced by spacing oil. The dilution channel may be upstream of the injection port as shown in FIG. 3.

The module may further comprise a sorter, the sorter for sorting droplets into a first droplet set and a second droplet set. The sorter may comprise a bifurcated sorting junction downstream of the picoinjector. Therefore, once the droplets have been picoinjected, the supply channel splits into two different channels: a first exit channel and a second exit channel. Mechanisms and ways to sort cell populations are set out above for the droplet generation module. These apply equally to the sorter incorporated into the picoinjection module.

The device shown in more detail in FIG. 3 has an injection point (“droplet re-injection”) to inject droplets into the supply channel. The supply channel has a droplet spacer (“spacing oil”). The device may additionally have a dilution channel (“diluting oil”). The dilution oil channel may also be between injection and the spacer.

Upon re-injection, the droplets may be diluted by the diluting oil in the diluting channel. This reduces merging of the droplets. The droplets are then spaced using the droplet spacer to allow evenly spaced droplets which maximises picoinjection efficiency.

After the droplets have been picoinjected the channel diameter may widen towards the exit to further prevent merging of the droplets.

The Third Microfluidic Device

The third microfluidic module may be a further picoinjector as shown in FIG. 3.

Additionally, the distance between the droplet spacer and the picoinjector may be modified to adapt to different sizes of droplet. For example, the droplet will be smaller in the second microfluidic device (as only the lysis and fragmentation reagent and bead have been added). Therefore, the droplets are less prone to merging compared to after additionally having the RNA tagging reagent added during the second step. To counteract this increased chance of merging in the third microfluidic device after the RNA tagging reagent has been added the distance between the droplet spacer and picoinjector can be increased as shown (FIG. 4b). FIG. 4a shows an example distance from the droplet spacer to the picoinjector for the second microfluidic device. The distance may be about 5 to 20 times the width of the channel. The distance in the third microfluidic device may then be approximately twice this: for example, to 40 times the width of the channel.

The reverse transcriptase may alternatively be added with the RNA tagging reagent in the second microfluidic device.

Alternatively, the third microfluidic device may be a droplet fusion module as shown in FIG. 6 (bottom right). Droplet fusion uses electro-coalescence at a fusion junction or chamber to merge droplets for example by applying an electric field to destabilize the droplets-oil interfaces. By fusion junction (or chamber) is meant the area in the module where the droplets meet and are coalesced.

By adding beads at the last modular step, the method generates smaller droplets after encapsulation. This increases the throughput and the method can proceed faster. By using smaller beads, the throughput can be increased even further.

The third device may comprise:
a) a fusion channel comprising a fusion chamber;
b) a bar-coded bead channel in fluid communication with the fusion channel for injecting beads;
c) a reverse transcriptase reagent channel in fluid communication with the fusion channel for injecting reverse transcriptase reagent; and
d) a droplet spacer in fluid communication with the fusion channel;
wherein the fusion chamber is downstream of channels b)-d).

The module comprises a fusion junction (or chamber) which is adapted to fuse the droplet comprising the tagged RNA, output from the second device (depicted as the small droplets in FIG. 23) and the droplet comprising the reverse transcriptase and bead (the larger droplets in the fusion junction in FIG. 23) together to form 1 droplet. In the fusion junction the tagged RNA droplet fuses with the RT and bead droplet. This may occurs due to electro-coalescence or various other known mechanisms.

The fusion chamber may be coupled to one or two electrodes which are configured to apply an electric field to the fusion chamber to electro-coalesce droplets in the fusion chamber. More detail is provided below.

The fusion junction may comprise a fusion channel also referred to here as a supply channel. The supply channel leads into the fusion junction. Microfluidic droplets comprising: a) the tagged RNA in a first droplet; and b) the reverse transcriptase and a bead in a second droplet, flow into the supply channel as shown in FIGS. 5 and 23.

Therefore, the module may further comprise: a channel in fluid communication with the supply channel into which droplets comprising tagged RNA as out-put from the second device may be introduced. These droplets then flow into the supply channel.

The droplet fusion module may comprise one or more further channels in fluid communication with the supply channel which flow microfluidic droplets comprising reverse transcriptase reagent and a bead into the supply channel and from there into the fusion junction. This is shown in FIG. 23.

The module may also comprise a droplet generation junction to form the droplets comprising the reverse transcriptase and bead. The left-hand side of this bottom figure shows a droplet generation junction in fluid connection with the supply channel. The droplet generation junction adapted to form microfluidic droplets comprising the bead and the reverse transcriptase mix. These droplets may then flow into the supply channel. The droplet generation junction may be in fluid communication with one or more channels which supply reverse transcriptase mix, beads and partitioning fluid into the junction to encapsulate the reverse transcriptase and bead in a microfluidic droplet. The droplet generation junction can use flow focusing, step emulsification or cross flowing (e.g. T-junction) droplet formation to form the droplets comprising reverse transcriptase and a bead. Alternatively, two separate droplets can be made, one having the reverse transcriptase reagent and the second having the bead.

The supply or fusion channel may not be necessary if the channel supplying the tagged RNA droplets and the channel supplying the droplet comprising the RNA and bead feed directly into the fusion chamber.

Droplets are fused in a droplet fusion module where synchronization of flowing droplets into the fusion channel allows droplet pairs to form (in a ratio of no more than 1 lysate droplet per 1 RT and bead droplet) followed by fusion in the fusion chamber.

The two electrodes may be salt electrodes filled with 5 M NaCl. However, other types of electrodes would be known to the skilled person in the art.

The current can be generated using a function generator and high voltage amplifier to continuously generate alternate current, for example at 250 V signal (peak-to-peak) and 10 kHz frequency, in order to cause droplet fusion in the fusion chamber.

The supply channel may comprise a channel with a width and or depth larger than the tagged RNA droplet but smaller than the RT/bead droplet. This helps the droplets group in pairs for fusion.

The droplet fusion module comprises a droplet spacer upstream of the fusion junction. The function of the spacer is to add spacer oil to evenly space the droplets prior to entry into the fusion junction. The droplet spacer may be downstream of the droplet generation junction. Alternatively or additionally, the droplet spacer may add spacer oil between the tagged RNA droplets. Therefore, the droplet spacer is in fluid communication with the channel which flows tagged RNA droplets into the supply channel. Alternatively or additionally, the droplet spacer may comprise an auxiliary channel in fluid communication with the supply channel wherein in use the auxiliary channel is attached to a reservoir of spacer oil

The droplet comprising the reverse transcriptase and bead may be approximately 1 nl in volume. For example, the droplet may be 0.1-5 nL, for example, 0.2-2 nL in volume. The size will be dependent on the bead used. The fusion of the tagged RNA droplet with the second droplet comprising the reverse transcriptase and bead results in a droplet which has a reverse transcriptase concentration (and optionally other reagent concentrations) as described above for example in Table 6.

As an alternative to a third device, the reverse transcriptase reagent can be added with the repair and polyadenylation reagent by picoinjection. After picoinjection of both reagents, different incubation temperatures can be used to perform the repair and polyadenylation and then the subsequent reverse transcription. For example, room temperature for 25 minutes, 8 minutes at 37° C., 50° C. for 2 hours and 70° C. for 20 minutes. Where two reverse transcriptases are used, additional temperatures can be added to allow both to work optimally, for example, room temperature for 25 minutes, 8 minutes at 37° C., 50° C. for 20 minutes (to denature polyA, to some degree), 42° C. for 1 hour, 50° C. for 30 minutes, ten cycles of 42° C. then 50° C. (2 minutes each) and 70° C. for 20 minutes. The same final concentrations in the droplet after picoinjection may be used as above for the repair and polyadenylation reagent and the reverse transcriptase reagent.

Alternatively, instead of adding the reverse transcriptase reagent using a third device, the reverse transcriptase can be added after de-emulsification. This can be done for example as follows:
1) Aspirate mineral oil from the tube
2) Aspirate surfactant oil from the tube
3) Ad 500 μl of 5×SSC buffer on top of the emulsions
4) Add 200 μl of 100% 1H,1H, 2H,2H-perfluoro-1-octanol
5) Spin-down for 1 minute at a 1000 g
6) Remove supernatant (˜450 μl of supernatant)
7) Wash once more with 500 μl of 5×SSC buffer (spin down and aspirate supernatant), and once with TET buffer, then spin down and remove all the supernatant.
8) Add the 500 μl of RT mix to the beads and incubate for 2 hours at 50° C.
9) Wash the beads twice with TET (10 mM Tris-HCl, 0.05% Tween and 0.5 mM EDTA).
10) Perform downstream exonucelase1 clean-up and wash twice with TET buffer
11) Perform second strand synthesis and IVT.

Proceed to standard downstream library preparation

Both of these alternative methods use the modular system of described above comprising a droplet generation module and picoinjection module.

Alternative Method without Fragmentation The methods comprising fragmentation allow for sequencing of the entire length of the RNA.

However, often sequencing of the ends of the RNA is sufficient for experimental needs (alternatively this method can be used where the RNA is already fragments as described above, for example is a poorly stored sample). A method is therefore provided which tags existing RNA allowing for the various types of RNA to be tagged and not only mRNA. The method is also modular, allowing for optimization of the individual steps as for the above methods. The method comprises:
a) encapsulating in a microfluidic droplet:
   i) a cell or cell structure comprising RNA; and
   ii) lysis and RNA tagging reagents, wherein the RNA tagging reagent adds an oligonucleotide tag to the RNA;
b) incubating the droplet to release the RNA from the cell or cell structure and to allow the RNA to be tagged with the oligonucleotide;
c) hybridizing the oligonucleotide tag of the RNA to a cDNA synthesis primer; and
d) performing reverse transcription to obtain a cDNA sequencing library wherein each cDNA in the cDNA sequencing library comprises a barcode.

The RNA tagging reagent may be a polyadenylation reagent and step e) allows polyadenylation; and the cDNA synthesis primer is a poly-T primer which hybridizes to the poly-A tag of the RNA.

The lysis reagent is as above, however, no MgCl₂ is added as there is no fragmentation in this method. The incubation after encapsulation would be room temperature (16-25° C.) for at least 20 minutes, for example 25 minutes, then a temperature of at least 50° C. for at least 20 minutes (to denature the thermolabile proteinase K).

If polyadenylation is used as the RNA tag, the RNA repair and polyadenylation reagent above may be used without the T4 PNK enzyme.

The method may additionally comprise a sorting step downstream of the encapsulation step which divides the droplets into a first droplet set and a second droplet set. The sorting may divide the droplets into those containing cell lysate from live cells (first droplet set) and those containing lysate from dead cells or empty droplets containing no cells (second droplet set).

The system described with a droplet generation module and picoinjection module as described above may be used to implement this method.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

### DESCRIPTION OF THE FIGURES

FIG. 1 shows the microfluidic and high-throughput VASA-seq workflow. VASA-seq refers to the previous non-microfluidic workflow described in WO2020/089218. VASA-drop refers to this method adapted for to a high throughput microfluidic workflow.

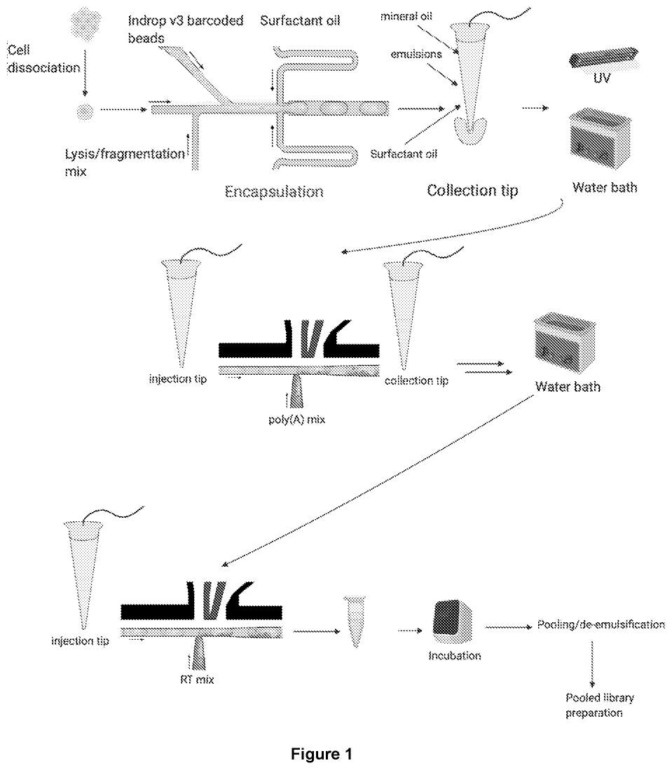

FIG. 2 shows the molecular process happening at each point in the microfluidic workflow.

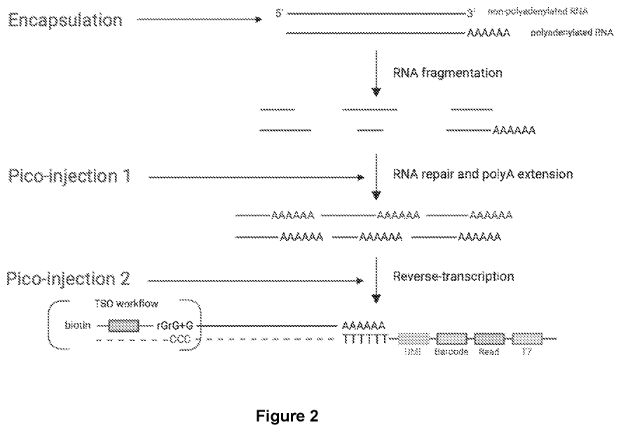

FIG. 3 shows the picoinjector module

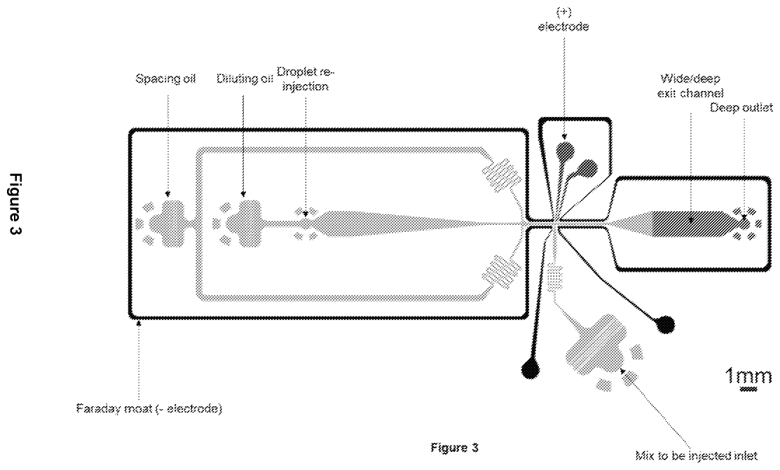

FIG. 4 shows the different picoinjector architectures used: FIG. 4a shows the picoinjector for the first injection of the repair and poly(A) mix (referred to as pico-injector A) and FIG. 4b shows the second picoinjector for the injection of RT mix (referred to as pico-injector B).

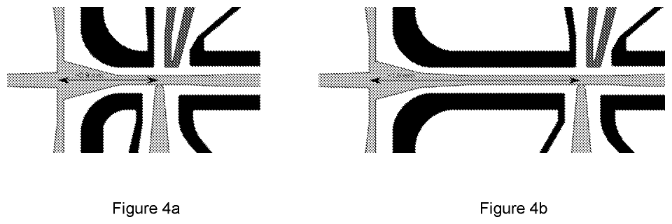

FIG. 5 shows an ultrahigh-throughput variation of the modular method using encapsulation and picoinjection for the first two steps then droplet fusion for the third step.

FIG. 6 shows the different modular devices of the high throughput device for implementing the method of FIG. 5 with a droplet fusion module as the third device. This system is referred to as SuperVASA.

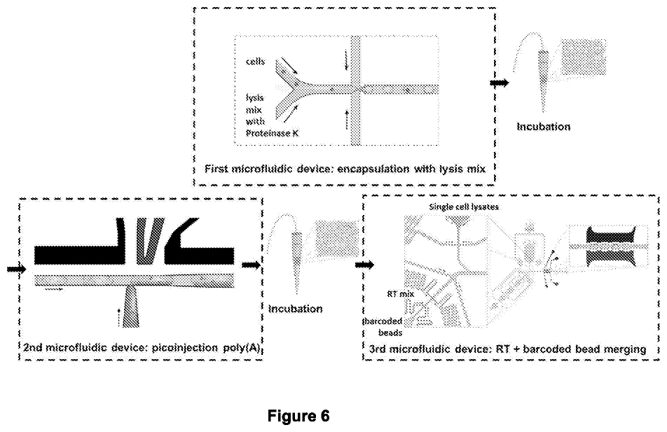

FIG. 7 a) shows number of genes per cell at different sequencing depths comparing droplet VASA-seq (VASA-drop) to other state-of-the-art methods b) shows 5′-to-3′ gene-body coverage for all methods. c) detection of different biotypes for all methods, with enhanced representation for sncRNAs. d) Percentages of unspliced reads detected as a distribution for the single-cells sequences for each method.

FIG. 8 shows mESc/HEK293T species mixing Quality Control; (Barnyard plot) depicting the number of UMIs detected, with a heterotypic doublets rate of 3.73%.

FIG. 9 shows E6.5, E7.5, E8.5 and E9.5 QC metrics (1/3). The figure shows the number of UMI (n_counts) molecules and genes (n_counts) detected per fraction of collected droplets (˜1,000 cells).

FIG. 10 shows E6.5, E7.5, E8.5 and E9.5 QC metrics (2/3). Dimensional reduction (UMAP) of the merged fractions for each stage illustrating great alignment between different fraction replicates, expected clusters and low doublet rates detected using Scrublet.

FIG. 11 shows E6.5, E7.5 E8.5 and E9.5 RNA velocity profiles projected on a dimensional reduction UMAP (3/3). Coverage of genes across their lengths allows for accurate estimation of unspliced to spliced ratios and more sensitive RNA velocity measurements.

FIG. 12 shows Gene-body coverage and splice-junction saturation profiling. The gene body coverage is for a set of fractions taken from all timepoints. The splice-junction saturation plot illustrates the known splice junctions discovered with increasing number of reads for median values of 10 random combinations of cells taken from the cells assigned to the epiblast at E6.5.

FIG. 13 shows: Alternative splicing pattern for the Lrrfipl gene discovered between Cardiomyocytes and Cardiomyocytes precursors extracted from E8.5 cells.

FIG. 14 shows the blocking of poly(A) extension on a TSO using a 3′ LNA locked and 5′ biotin blocked TSO.

FIG. 15 shows an overview of the early organogenesis atlas projected on a UMAP encompassing timepoints E9.5, E10.5 and E11.5, generated using the superVASA workflow.

FIG. 16 shows the distribution of the number of genes detected for cells sequenced at each timepoint (E9.5, E10.5, E11.5)

FIG. 17 shows cell-type annotation using markers for each Leiden cluster of a fraction of the cells from the E11.5 timepoint, projected on a UMAP dimensional reduction.

FIG. 18 shows the 5′ to 3′ gene body coverage for protein coding gene using the E11.5 timepoint sequencing reads as an input to the RSeQC tool.

FIG. 19 shows bioanalyzer traces (high-sensitivity kit) of the final pooled library representative of different DNA purification methods and the depletion after ligation optimized protocol, showing an effective depletion of fragmented rRNA peaks in the final library.

FIG. 20 shows: a) is a schematic of the single aqueous inlet used for droplet generation of PAAm droplets. b) shows an illustration of the triple bead barcoding process utilised in the superVASA process. c) proposes an overview of the molecular steps involved in the whole-transcriptome barcoding process, with the RNaseH depletion steps interchanged with the adapter ligation steps.

FIG. 21 shows schematic of the microfluidic device used for the first step of superVASA protocol: generation of droplets stained with Calcein and next sorting of droplets containing 1 cell. Sorting is performed by a dual-fibre system that detects fluorescence and triggers a dielectrophoretic sorting of droplets with 1 cell into the positive channel. Numbers 1-7) indicate inlet channels for following liquids: 1) cell suspension, 2) lysis mix, 3) carrier oil for droplet formation, 4) spacing oil, 5) bias oil facilitating sorting, 6) (optional) carrier oil for generation of buffer droplets, 7) (optional) buffer aqueous solution for generation of buffer droplets. Number 8) indicates outlet for droplets with 1 cell and optionally buffer droplets and number 9) indicates outlet for waste droplets.

FIG. 22 shows schematic of the pico-injector used for injecting the poly(A) and RNA repair mixture in the droplets containing lysates of single cell or single cell structure. Numbers 1-4 depict inlet channels for following liquids: 1) spacer oil, 2) dilution oil for making droplet emulsion less densely packed, 3) droplet emulsion, 4) pico-injection liquid of poly(A) and RNA repair mix. Number 5) depicts outlet for droplets.

FIG. 23 shows schematic of the droplet fusion device used to merge a droplet with a bead and the reverse transcriptase with the droplets comprising tagged RNA. The synchronized and paired droplets are then merged using electric field provided by salt electrodes. Numbers 1-6 depict inlet channels for following liquids: 1) suspension with densely packed beads, 2) reverse transcriptase mix 3) carrier oil for droplet generation, 4) spacer oil for droplets with RT and a bead, 5) spacer oil for single-cell lysate droplets 6) single-cell lysate droplet emulsion. Number 7) depicts outlet for droplets.

### EXAMPLES

Aspects of the present invention will now be illustrated by way of example only and with reference to the following experimentation.

As a guide, concentration of the various reagents during the method are provided below for:
Table 7: the method where the bead is added with the lysis reagent (claim 5: referred to as “VASA-drop” below); and
Table 8: the method where the bead is added with the reverse transcriptase by droplet fusion (claim 10: referred to as “superVASA” when sorting is additionally used after the encapsulation step to sort droplets comprising live cells from droplets comprising dead cells and/or droplets comprising more than 1 cell or cell structure). That is, the superVASA device is comprises the device of claim 24b with the addition of a sorter after droplet encapsulation.

For clarity, VASAdrop is the workflow of methods of claims 1 and 8; implemented by the device of claims 22 and 24a.

SuperVASA is the workflow of methods 1, 9 and 20a; implemented by the device of claims 22, 24b and 28a.

TABLE 7
Bead added with lysis reagent

| | | | | [symbol] EPA | Thermolab | | | | |
|---|---|---|---|---|---|---|---|---|---|
| | [symbol] | KCl | MgCl [symbol] | L- [symbol] | [symbol] | dNTP [symbol] | PB [symbol] | Optpr [symbol] | EDTA |
| [symbol] | 2 [symbol] | 2 [symbol] | 14.2 [symbol] | [symbol] | [symbol] | 0.5 [symbol] | 0 | 0 | 0 |
| in mix | | | | | | | | | |
| [symbol] | 0 | 0 | 0 | 0 | 0 | 0 | [symbol] | [symbol] | 0 |
| [symbol] | [symbol] | [symbol] | [symbol] | 0. [symbol] | 0 | 0 | 0 | 0 | 0.0 [symbol] |
| Bead [symbol] | | | | | | | | | |
| [symbol] | [symbol] | [symbol] | [symbol] | 0.2% | 2 [symbol] | 0.1 [symbol] | 0. [symbol] | [symbol] | 0.0 [symbol] |
| Final | | | | | | | | | |
| droplet | | | | | | | | | |
| after [symbol] | | | | | | | | | |
| [symbol] | 6 [symbol] 1 | 6 [symbol] 2 [symbol] | 2. [symbol] | 0 | 0 | 0 | 0 | 0 | 0 |
| [symbol] | 8 [symbol] | [symbol] | 4. [symbol] | 0.1 [symbol] | 1.5 [symbol] | 0.12 [symbol] | 0.2 [symbol] | 4.2% | 0.01 [symbol] |
| droplet | | | | | | | | | |
| after [symbol] | | | | | | | | | |
| [symbol] | 62 [symbol] mM | [symbol] | 2.3 [symbol] | 0 | 0 | 1 [symbol] | 0 | 0 | 0 |
| RT mix | | | | | | | | | |
| [symbol] | 73 [symbol] | 76.6 [symbol] | [symbol] | 0.07% | 0.7 [symbol] | 0.6 [symbol] | 0.1 [symbol] | 1. [symbol] | 0.00 [symbol] |
| droplet | | | | | | | | | |
| after [symbol] | | | | | | | | | |

| | | | | | E. coli | | |
|---|---|---|---|---|---|---|---|
| | | | | T4 | polA | | Superscript |
| | Tween 20 | DTT | ATP | PNK | polymerase | Rna [symbol] | [symbol] RT |
| [symbol] | 0 | 0 | 0 | 0 | 0 | 0 | 0 |
| in mix | | | | | | | |
| [symbol] | 0 | 0 | 0 | 0 | 0 | 0 | 0 |
| [symbol] | 0.0 [symbol] | 0 | 0 | 0 | 0 | 0 | 0 |
| Bead [symbol] | | | | | | | |
| [symbol] | 0.01 [symbol] | 0 | 0 | 0 | 0 | 0 | 0 |
| Final | | | | | | | |
| droplet | | | | | | | |
| after [symbol] | | | | | | | |
| [symbol] | 0 | 15. [symbol] | 0. [symbol] | [symbol] 1 [symbol] | 2 [symbol] | [symbol] | 0 |
| [symbol] | 0.01% | 4 [symbol] | 0. [symbol] | [symbol] | 62. [symbol] | [symbol] | 0 |
| droplet | | | | | | | |
| after [symbol] | | | | | | | |
| [symbol] | 0 | [symbol] | 0. [symbol] | 0 | 0 | 1.2 [symbol] | [symbol] |
| RT mix | | | | | | | |
| [symbol] | 0.00 [symbol] | 6.1 [symbol] | 0.022 [symbol] | 3 [symbol] | 2 [symbol] | 9 [symbol] | [symbol] |
| droplet | | | | | | | |
| after [symbol] | | | | | | | |

[symbol]

indicates data missing or illegible when filed

TABLE 8
Bead added by droplet fusion

| | [symbol] | KCl | N [symbol] Cl | MgCL2 | [symbol] | Th [symbol] | [symbol] | PB [symbol] | Op [symbol] | BSA | EDTA |
|---|---|---|---|---|---|---|---|---|---|---|---|
| [symbol] | 14 [symbol] | [symbol] | | [symbol] | 0. [symbol] | [symbol] | 0. [symbol] | 0 | 0 | 0 | 0 |
| in mix | | | | | | | | | | | |
| [symbol] | 0 | 0 | 0 | 0 | 0. [symbol] | 0 | 0 | [symbol] | [symbol] | 0.04% | 0 |
| [symbol] | [symbol] | [symbol] | 0 | [symbol] | 0.2 [symbol] | [symbol] | 0.2 [symbol] | 0. [symbol] | 7. [symbol] | 0.02% | 0 |
| [symbol] | [symbol] | [symbol] | 0 | [symbol] | 0 | 0 | 0 | 0 | 0 | 0 | 0 |
| [symbol] | [symbol] | [symbol] | 0 | [symbol] | 0. [symbol] | 2. [symbol] | 0. [symbol] | 0. [symbol] | [symbol] | 0.01 [symbol] | 0 |
| [symbol] | [symbol] | 0 | [symbol] | [symbol] | 0 | 0 | 0.3 [symbol] | 0 | 0 | 0 | 0 |
| bead | [symbol] | [symbol] | 0 | 3 [symbol] | 0. [symbol] | 0 | 0 | 0 | 0 | 0 | 0.0 [symbol] |
| mix | | | | | | | | | | | |
| final | [symbol] | [symbol] | [symbol] | [symbol] | 0.0 [symbol] | [symbol] 0 [symbol] | 0. [symbol] | 0 | 0 | 0 | 0.00 [symbol] |
| mix | | | | | | | | | | | |
| after | | | | | | | | | | | |
| second [symbol] | | | | | | | | | | | |

| | | | | | E. coli | | | | |
|---|---|---|---|---|---|---|---|---|---|
| | Tween | | | | polyA | | Superscript | Max | TSD |
| | 20 | DTT | AT [symbol] | T4 [symbol] | polymerase | [symbol] | [symbol] RT | [symbol] | [symbol] |
| [symbol] | 0 | 0 | 0 | 0 | 0 | 0 | 0 | 0 | 0 |
| in mix | | | | | | | | | |
| [symbol] | 0 | 0 | 0 | 0 | 0 | 0 | 0 | 0 | 0 |
| [symbol] | 0 | 0 | 0 | 0 | 0 | 0 | 0 | 0 | 0 |
| [symbol] | 0 | [symbol] | [symbol] | [symbol] | [symbol] | [symbol] | 0 | 0 | 0 |
| [symbol] | 0 | [symbol] | 0.0 [symbol] | [symbol] | [symbol] | [symbol] | 0 | 0 | 0 |
| [symbol] | 0 | [symbol] | 0 | 0 | 0 | [symbol] | [symbol] | [symbol] | [symbol] |
| bead | 0.0 [symbol] | 0 | 0 | 0 | 0 | 0 | 0 | 0 | 0 |
| mix | | | | | | | | | |
| final | 0.00 [symbol] | [symbol] | 0 | 0 | 0 | [symbol] | [symbol] | [symbol] | [symbol] |
| mix | | | | | | | | | |
| after | | | | | | | | | |
| second [symbol] | | | | | | | | | |

[symbol]

indicates data missing or illegible when filed

### Example 1: Method Described Extracts More RNA from Cells than Current Methods

Species mixing experiment with VASA-seq using mouse embryonic stem cells and human HEK293T cells.

a) Cell Harvesting

HEK293 Ts were passaged every second day and cultured in T75 flasks. The culture media was DMEM (4500 mg/L gluc & L-glut & Na bicarb, w/o Na pyr, D5796-500 ML, Sigma) supplemented with 10% heat-inactivated FBS and 1× Penicillin-Streptomycin. For passaging and collection, the cells were washed with 10 ml of ice-cold 1× PBS (Lonza) twice. 9 ml of PBS was added to the flask and cells were detached by adding 1 ml of 10×Trypsin-EDTA (Sigma-Aldrich) and incubated at 37° C. for 5 minutes. Trypsin-EDTA was then inactivated with 15 ml of DMEM 10% FBS and incubated at 37° C. for 5 minutes.

Mouse embryonic stem cells (mESc) were cultured in 2i+LIF (Dulbecco's Modified Eagle Medium F-12 (DMEM/F-12) Nutrient Mixture, without L-Glutamine and Neurobasal Medium without L-Glutamine in a 1:1 ratio, 0.1% Sodium Bicarbonate (7.5%), 0.1% Bovine Albumin Fraction V Solution (7.5%), 0.5× B-27 Supplement (50×), 0.5× N-2 Supplement (100×), 0.1 mM 2-Mercaptoethanol (50 mM), 2.2 nM L-Glutamine (200 nM), 110 U/ml Penicillin-Streptomycin (10,000 U/ml), 20 μg/ml Insulin Zinc (4 mg/ml), 0.2 μg/ml mLIF (10 μg/ml), 3 μM CHIRON99021 (10 mM), 1 μM PD0325901 (10 mM)). The culture supernatant was aspirated and 500 ul Accutase per 5 ml of culture was added for cell dissociation. After 5 minutes of incubation at 37C, 4.5 ml of wash buffer was added (Dulbecco's Modified Eagle Medium F-12 (DMEM/F-12) Nutrient Mixture, without L-Glutamine, 1% Bovine Albumin Fraction V Solution (7.5%)).

The cells were then separately pelleted at 300 g for 3 minutes and the supernatant was aspirated. The cells were washed three times in 1× PBS, and brought to a concentration of 250 cells per μl (each, 500 cell per μl total). The cells were then mixed 1:1 with a solution of 1× PBS+30% (v/v) Optiprep to constitute the cell mix. The lysis mix was made fresh before each encapsulation, as follows: 0.5 mM dNTPs (ThermoFisher, 10 mM), 0.52% IGEPAL-CA630 (Sigma-Aldrich, 100%), 40 mM Ultrapure Tris-HCl ph 8 (Life sciences, 1M), 3.76× First Strand Buffer (Invitrogen, 5×), 3 mM Magnesium Chloride (Ambion, 1M) and 6 U/ml thermolabile proteinase K (NEB, 120 U/ml). The barcoded polyacrylamide beads were prepared as previously described (https://doi.org/10.1038/nprot.2016.154, Nature protocols). The three suspensions were loaded in the tubing of three individual 1 ml SGE glass syringes filled with PBS. The injection flow rates for the droplet encapsulation device were: the cell suspension was flown at 85 μl/hr, the bead suspension was flown at 65 μl/hr, the lysis solution was flown at 75 μl/hr and the 5% RAN in HFE7500 surfactant was flown at 450 μl/hr. The average droplet size was ˜0.55 nl for these flow rates and a microfluidic device depth of 80 urn. The device was primed as previously explained (Zilionis et al., https://doi.org/10.1038/nprot.2016.154) and the droplets were collected for approximately one hour in a 1 ml pipette tip pre-filled with mineral oil and connected to a tubing via a PDMS connector. The collection tip was then closed using a 1 ml SGE glass syringe pre-filled with mineral oil and connected to a glass bonded PDMS plug.

a. Cell Lysis and RNA Fragmentation

The tip container was further left at room temperature (23° C.) for 20 minutes to allow for cell lysis to occur and the tip was further placed in a container surrounded by aluminium foil (see Extended methods) and the barcodes were photocleaved off using a High-Intensity UV Inspection Lamp (UVP) that was switched on for 7 minutes. The container was then further submerged in a water bath at 85° C. for 6 minutes and 30 seconds. The container was then immediately submerged in an ice bucket filled up with half proportions of ice and water.

b. RNA Repair and polyA Tailing

The droplets were re-injected in pico-injector and coalescence-induced merging with a poly(A) solution consisting of 26.6 mM Tris-HCl pH 8 (Invitrogen, 1M), 15.8 mM DTT (Invitrogen, 0.1M), 0.83× First Strand buffer (Invitrogen, 5×), 0.19 mM ATP (NEB, 10 mM), 3.15 kU/ml T4 Polynucleotide kinase (NEB, 10 k U/ml), 250 U/ml E. coli poly(A) polymerase, 2.6 kU/ml RNaseOUT (Applied biosystems, 40 kU/ml). The droplets were spaced in a first instance before re-injection in a flow-focusing junction using 5% RAN in HFE7500. The merging was applied by prefilling the electrode section of the device with 5M NaCl as previously described (Sciambi et al. https://doi.org/10.1039/C4 LC00078A). The function generator (TG2000, AIM-TTi) was used to generate square waves of amplitude 2.5 V and 10 kHz frequency, which was further amplified 100 times by the high-voltage power amplifier (Trek 601-C) and delivered to the merging junction on a chip via aqueous salt electrodes. The flow rates used were 200 μl/hr for the droplets, 60 μl/hr for the poly(A) mix, 120 μl/hr for the first spacing oil and 400 μl/hr for the second spacing oil. This generated ˜0.8 nl droplets at 70 Hz. The droplets were collected in a 1 ml collection tip and incubated for 25 minutes at room temperature (23° C.) followed by 8 minutes in a 37° C. water bath. The collection tip was then submerged in an ice-cold water bath for 2 minutes. The droplets were immediately processed for reverse-transcription after that.

c. Reverse Transcription

The droplets were re-injected in pico-injector B similarly to the previous step, albeit the injected droplets were collected in fractions of ˜1000 cells (˜27 μl of loaded droplets) in 1 ml LoBind Eppendorf tubes pre-filled with 200 μl of mineral oil. The droplets were injected with a reverse transcription mix constituted of 25 mM Tris-HCl ph8 (Invitrogen, 1M), 8 mM DTT (Invitrogen, 0.1M), 0.75× First Strand buffer (Invitrogen, 5×), 1 mM dNTPs, 20 kU/ml Superscript III (Invitrogen, 200 kU/ml), 1.2 kU/ml RNAseOUT (40 kU/ml). The flow rates for this device were as follows: 70 μl/hr for the first spacing oil, 700 μl/hr for the second spacing oil, 300 μl/hr for the re-injected droplets and 255 μl/hr for the RT mix. The collected fractions were incubated at 50° C. for 2 hours and then heat-inactivated at 70° C. for 20 minutes. For de-emulsification of the droplets, the mineral oil and oil phase were aspirated using a gel tip (Corning). Then 500 μl of filtered HFE7500 was added to the emulsions, followed by 500 μl of 100% 1H,1H, 2H,2H-perfluoro-1-octanol. The tubes were centrifuged for 5 second on a tabletop centrifuge, then 800 μl of the oil phase was removed and 500 μl of fresh HFE7500 oil was added. At this point the fractions were stored at −80° C. The downstream library preparation was achieved according to the procedure described in the VASA-seq patent application (WO2020/089218).

d) Bioinformatic Processing of the Sequenced Libraries for Quality Control

VASA-drop and published Smart-seq3 and 10× v2 libraries from HEK293T were demultiplexed, quality controlled using FastQC and mapped using the STAR aligner using the GRCh38 genome and ensemble v99 annotations. Further custom scripts were used to assign the reads for small non-coding RNAs. The count matrices were generated and imported into Rstudio where further tertiary analysis was performed.

e) Bioinformatic Processing of the Sequenced Libraries for the Species-Mixing Experiment

Mouse ES cells and HEK293T were re-suspended at equal amounts in the cell suspension buffer and ran through the Vasa-seq workflow in droplets as previously described. The libraries were sequenced, demultiplexed using Pheniqs and quality controlled using FastQC. The zUMIs pipeline was then used for mapping and counting on a concatenated GRCh38 and GRCm38 genome, using ensemble v99 annotations. Further downstream processing was achieved in Rstudio.

Results:

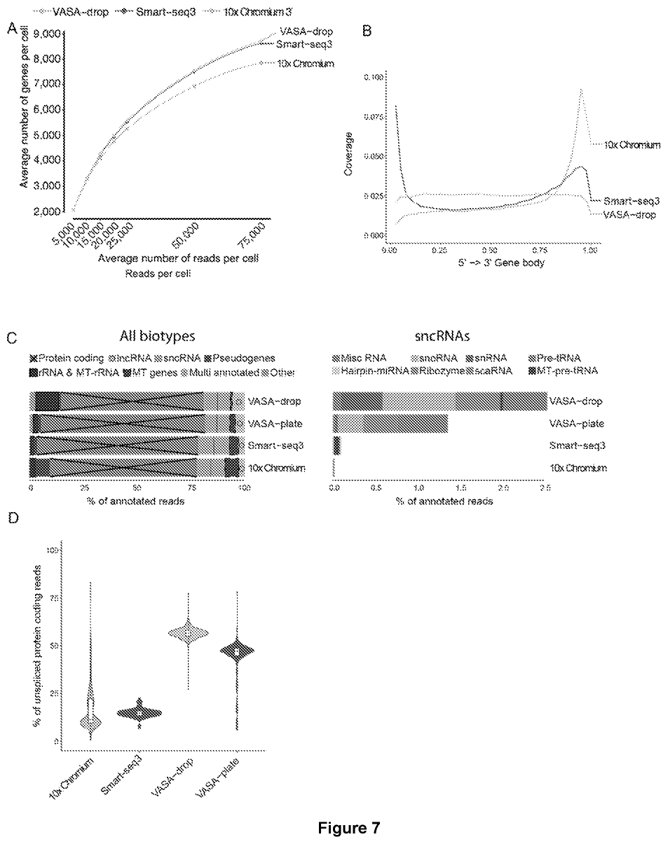

FIG. 7 shows the comparison between VASA-drop, Smart-seq3 and 10× for HEK293T cells. VASA-drop and Smart-seq3 libraries exhibited similar number of genes detected per cell whilst 10× detected fewer genes per cell (FIG. 7a). The gene body coverage showed that VASA-drop has an even detection from 5′-to-3′ while Smart-seq3 had a large 5′-bias and a smaller 3′-bias. Most reads for 10× data were stacked at the 3′-end (FIG. 7b). Protein coding fragments was the most abundant biotype in all methods, but VASA-drop detected approximately twice as many lncRNA molecules compared to Smart-seq3 and 10×. Only VASA-drop detected a significant amount of small ncRNA (sncRNA) (FIG. 7c). For VASA-drop, the majority of the reads detected were unspliced, whilst the vast majority for Smart-seq3 and 10× comprised spliced transcripts (FIG. 7d).

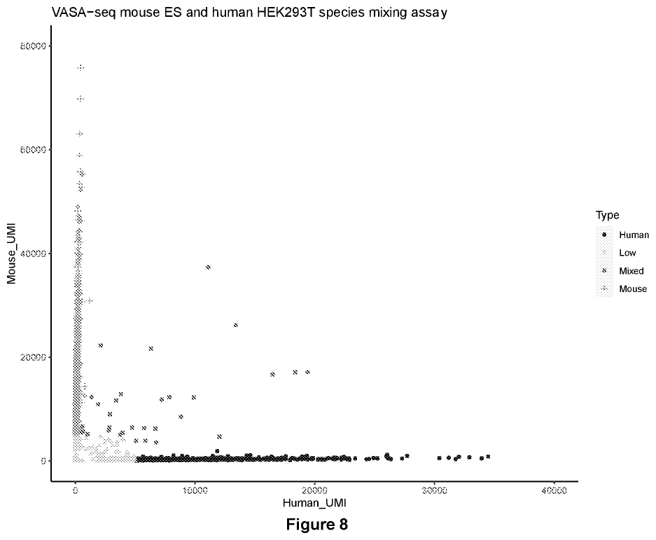

FIG. 8 depicts a species mixing assay for VASA-drop using mouse ES and human HEK293T cells as an input. The detected heterotypic rate was 3.73%, illustrating the retention of the single-cell lysate compartmentalization throughout the VASA-seq droplet workflow.

### Example 2: Single-Cell Total RNA-Seq Profiling of Gastrulating Mouse Embryos

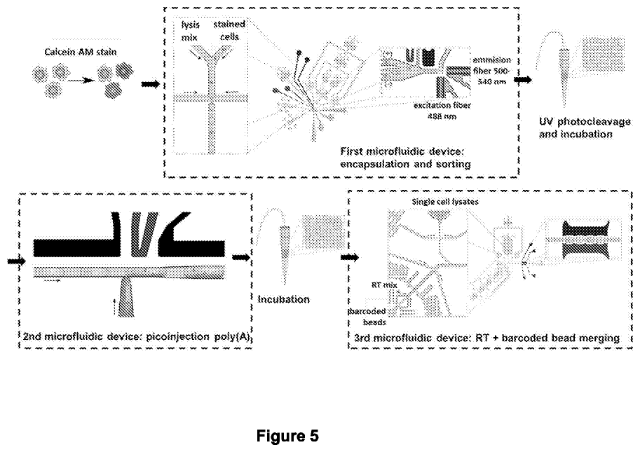

A) Embryo harvesting and single-cell suspension generation Pregnant C57BL/6 female mice (mated at 7 weeks of age) were purchased from Charles River or obtained from natural mating of C57BL/6 mice (Charles River) in house. Mice were maintained on a lighting regime of 14:10 hours light:dark with food and water supplied ad libitum. Detection of a copulation plug following natural mating indicated embryonic day (E) 0.5. Following euthanasia of the females using cervical dislocation, the uteri were collected into PBS with 2% heat-inactivated FCS and the embryos were immediately dissected and processed for scRNA-seq. Mouse embryos were dissected at time points E6.5, E7.5 E8.5 and E9.5 as previously reported. Since development can proceed at different speeds between embryos, even within the same litter, careful staging by morphology was adopted to exclude clear outliers. Embryos from the same stage were pooled into a low binding tube (Eppendorf, LoBind). E8.5 and E9.5 embryos were cut into pieces under the microscopy before imaging (FIG. 5) and collecting into a tube. The pooled sample was centrifuged at 300 g for 5 min at 4° C. The supernatant was aspirated and 100-200 μl of TrypLE Express dissociation reagent (Life Technologies). The tube was incubated at 37° C. for 7 min in a shaker. For the quench reaction, 1 ml of 30% FBS was added to the tube. The resulting single-cell suspension was washed with PBS and resuspended in PBS with 0.4% BSA and filtered through a Flowmi Tip Strainer with 40-μm porosity (ThermoFisher Scientific, 136800040). The cells were then processed similarly to the mouse ES and human HEK293T species mixing experiment, except the cells were sequenced on a Novaseq 6000 S2 platform. The resulting libraries were de-multiplexed using Pheniqs and the reads were mapped and counted using the zUMIs pipelines. Downstream tertiary analysis was performed using Scanpy, Scrublet and scVelo for quality scores, doublet detection, Leiden duster identification and plotting of RNA velocities.

Results:

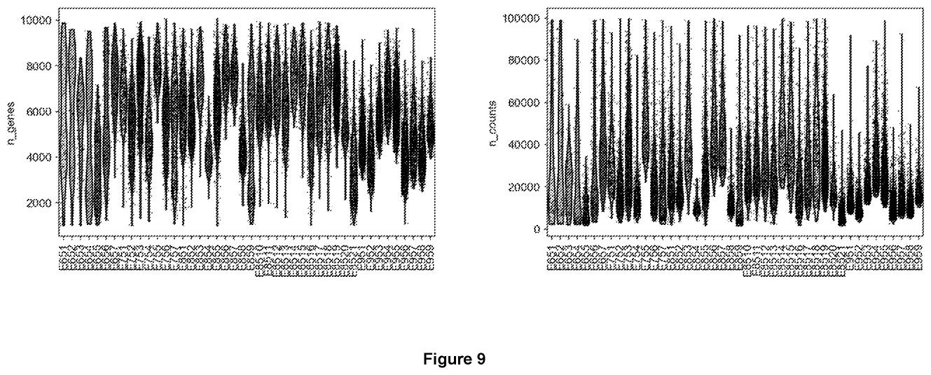

FIG. 9 Illustrates the number of UMIs (n_counts) and Genes (n_genes) detected for each pool in the samples, showing high number of unique molecules identified between separate fractions of pooled droplets.

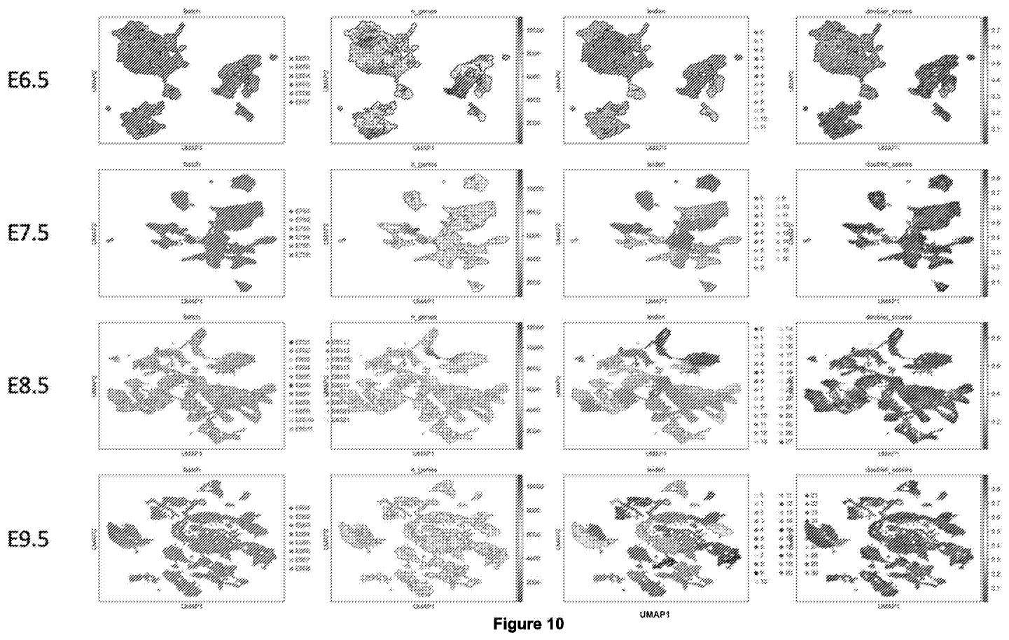

FIG. 10 shows the cells after dimensional reduction, leiden clustering and droplet doublet estimation using Scrublet. The cell types discovered for each stage overall match expected output from previous sequencing efforts using 3′ scRNA-seq. Each fraction of droplets collected throughout the process align perfectly and show good reproducibility. Furthermore, the low doublet detection rate illustrates the overall success in maintaining the compartmentalization of the droplet after cell lysis.

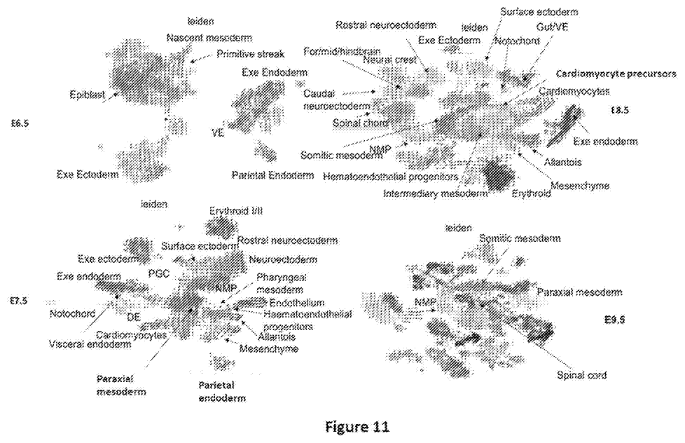

FIG. 11 illustrates the velocity profiles at each stage of the mouse embryo developmental process, showing increased granularity compared to published dataset for developmental events such as primitive streak formation (E6.5), Cardiomyocytes and endothelium formation (E7.5), somitogenesis and heart field formation (E8.5 and E9.5).

### Example 3: Detection of Alternative Splicing in the Mouse Gastrulation Dataset

The deduplicated bam files produced by the zUMIs pipeline were then used as an input for gene body coverage detection using the RSeQC′d geneBody_coverage.py function for different fractions. The bam files for the cells from the epiblast at E6.5 were then merged into ten fractions of 1, 2, 5, 10 and 20 and the detection rates of known splice junctions was computed using the junctionsaturation.py function from the RSeQC package, and the resulting median values of all ten comparisons was further computed. All bam files were demultiplexed into single-cell bam files and the cluster annotations obtained from the Leiden clustering with Scanpy were used for pairwise comparison of alternative splicing motives using the microExonator pipeline with different amount of cells per combination to determine the PSI value for detection of new AS patterns.

Results:

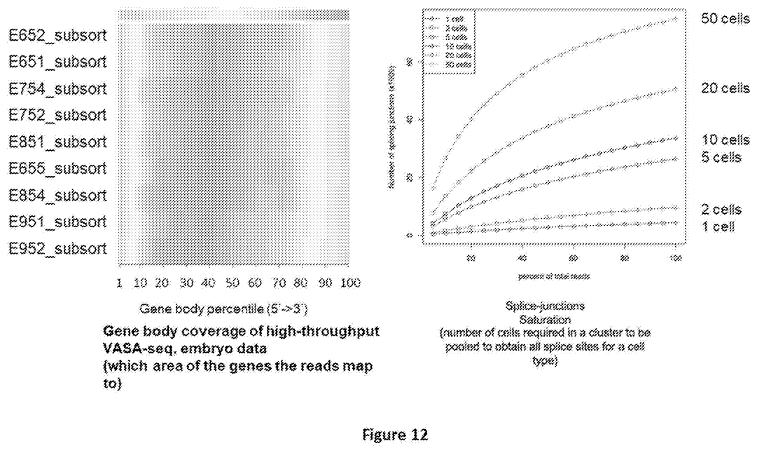

FIG. 12(left) Shows the gene body coverage for duplicate fractions at each timepoint E6.5, E7.5, E8.5 and E9.5, illustrating that the majority of reads map to the gene across its' body (5′ to 3′)

FIG. 12(right) shows the amount of detected known splice junctions using ten random permutations of pooled epiblast cells from E6.5, sequenced at a depth of 50 k read per cell. Noticeable saturation in the detection of known splice junctions for the median of 10 random permutations of 50 cells.

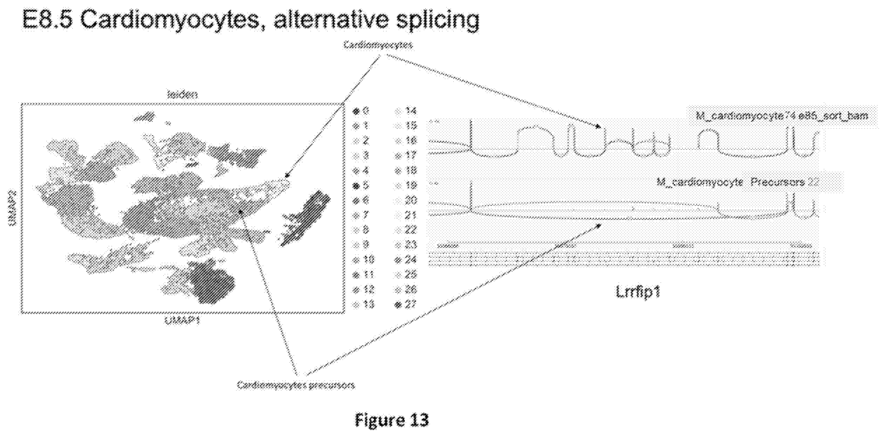

FIG. 13 Illustrates one of the alternatively splicing patterns identified for the gene Lrrfipl by pooling cells from the Cardiomyocytes against the Cardiomyocytes precursors at E8.5 and detecting splicing. The splicing pattern identified in the mesenchyme is an alternative exon pattern with no annotation, meaning the gene is highly alternatively spliced in existing datasets.

### Example 4: Usage of Template Switching Oligonucleotides with the E. coli Poly(A) Enzyme

2 uM of 3′ LNA-blocked and non-LNA blocked TSO were mixed with 1× E. coli poly(A) polymerase reaction buffer and 28.4 U/ml E. coli poly(A) polymerase and 0.2 mM ATP for 30 minutes at 37° C. The reaction mix was then diluted 1:100 in nuclease-free water and ran on a Bioanalyzer HS kit.

Results:

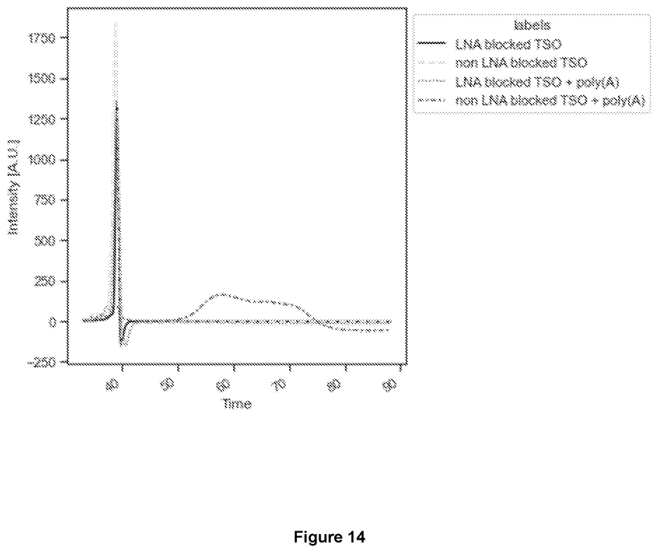

FIG. 14 illustrates the ability of 3′ LNA-blocked TSO to block poly(A) extension, which enables the use of TSO oligonucleotides in the VASA-seq droplet microfluidic protocol.

### Example 5: Profiling of 300 k Single Cells from Mouse Organogenesis (E9.5 to E11.5)

Mouse embryos from three different timepoints were sequenced (E9.5, E10.5 and E11.5) using superVASA as a follow-up from the VASA-seq study. The study encompasses ˜300 k single-cell total RNA-seq transcriptomes that are now being compared to another scalable method (sci-RNA-seq3).

superVASA protocol comprises 3 steps:
1) Encapsulation and sorter
2) Picoinjection of polyA mix
3) Fusion with droplet with RT/bead

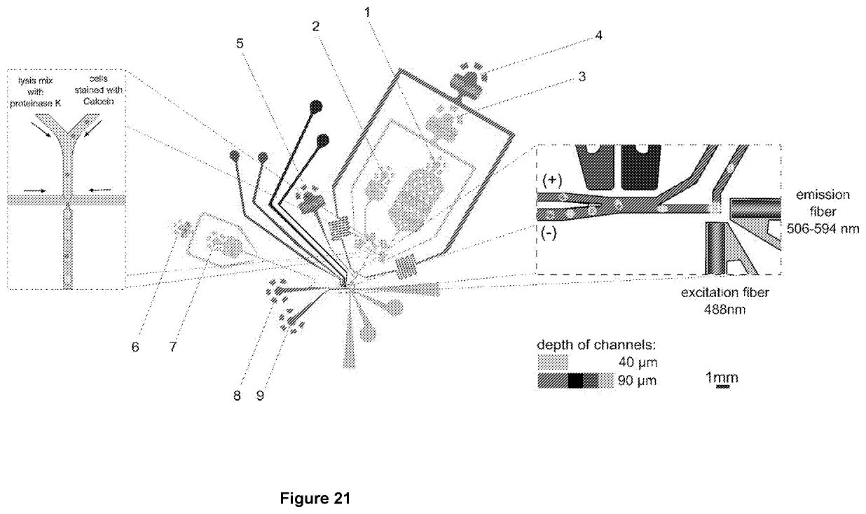

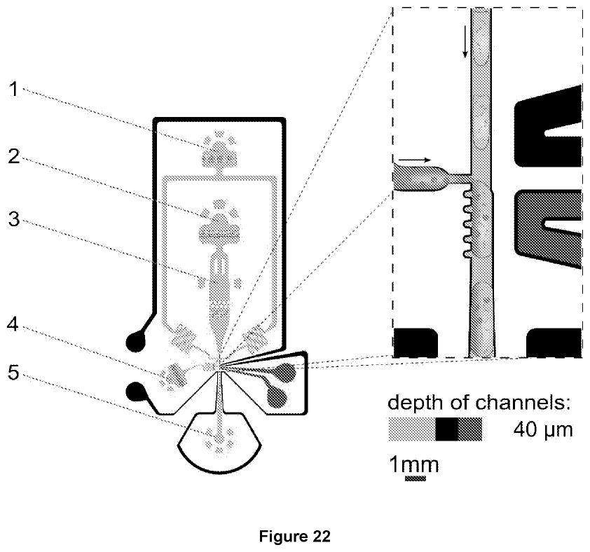

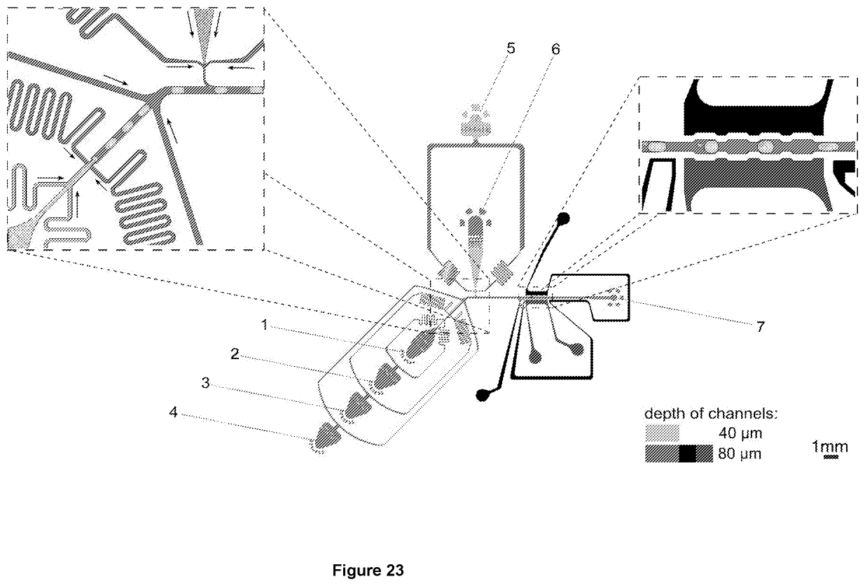

Murine embryo collection and sample pre-processing was performed similarly to stages E8.5 and E9.5 for the VASA-seq workflow but applied to stages E9.5, E10.5 and E11.5 (cutting into smaller pieces followed by dissociation with TrypLE and cell straining). Triple barcoding of PAAm beads was achieved as for the inDrop protocol, but with an intermediary oligonucleotide barcode extension step to increase the total barcode diversity to 14,155,776. The third barcoding step and enzymatic digestion was achieved as for inDrop but the last oligonucleotide sequence was changed to account for the intermediary overhang introduced by the method. The superVASA protocol uses the reaction mixes described in Table 8 for each step. The loading cell concentration for the cell containing solution used as an input for encapsulation was 5 M/ml (in 1× PBS, 15% Optiprep, 0.05% BSA). The cell and lysis flow rates for the encapsulation process were 120 μl/hr. The carrier oil phase was flown at 1,450 μl/hr which allowed for the generation of 28 μl droplets. The pico-injection step was miniaturized from the VASA-seq workflow to accommodate for the decrease in droplet size. This workflow is shown in FIGS. 21-23. The droplets were flown at 50 μl/hr, the diluting oil was flown at 10 μl/hr, the poly(A) tailing mix was flown at 16.6 μl/hr and the spacing oil was flown at 200 μl/hr. For droplet merging, the beads were prepared as for VASA-seq and flown in the droplet merging device at 90 μl/hr and the RT mix was flown at 350 μl/hr (25 mM Tris-HCl pH 8, 30 mM NaCl, 10 mM OTT final, 0.25 mM dNTPs (each), DTT (Invitrogen, 0.1M), 0.75× First Strand buffer (Invitrogen, 5×), 1 mM dNTPs, 20 kU/ml Superscript III (Invitrogen, 200 kU/ml), 1.2 kU/ml RNAseOUT (40 kU/ml)). The remainder of the library preparation was similar to VASA-seq, although the adapter ligation and rRNA depletion steps were inverted in their order (described in example 6). The final product was amplified using a dual-indexed PCR primer pair to minimize index hopping containing the P5 and P7 flow-cell adapters as overhangs. The libraries were sequenced as follows: 133 cycles for Read1, 31 cycles for i7, 8 cycles for i5, 44 cycles for Read2. The dataset was pre-processed as for VASA-seq, and tertiary analysis was performed using Scanpy and Seurat. The gene body coverage plot was achieved using the RSeQC package with the geneBody_coverage.py function.

Results:

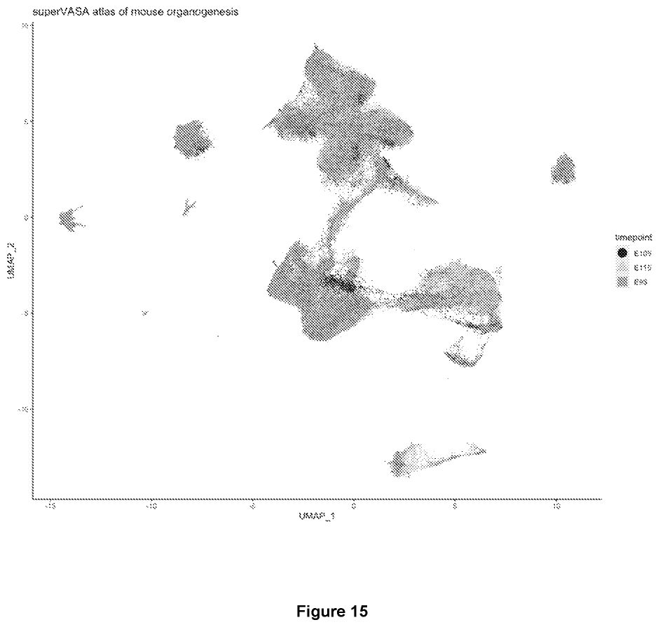

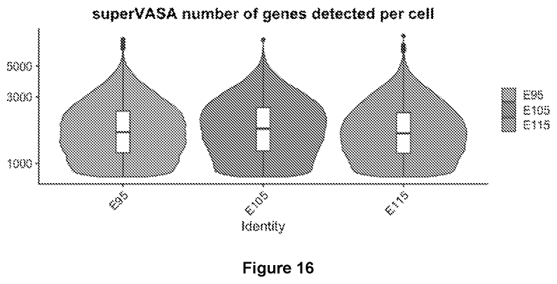

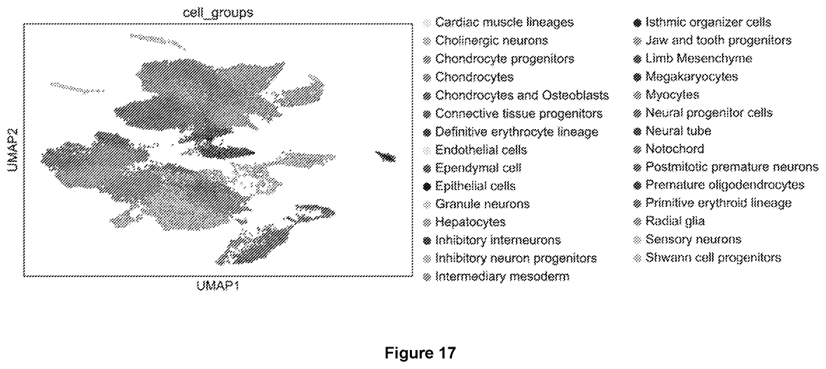

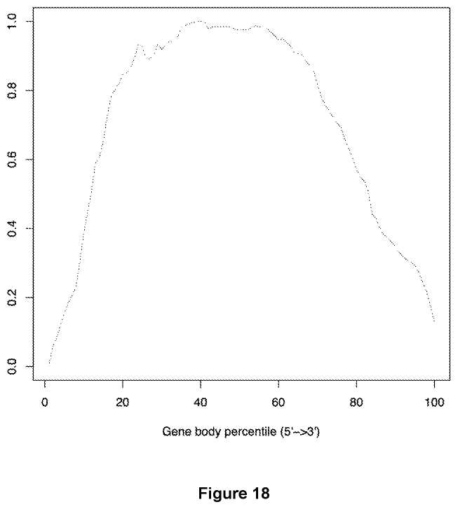

The method shows a representation of all cell types encompassing mouse early organogenesis, with no selection of specific cell-types due to the Calcein-AM staining of cells. The full atlas encompassing the 300 k cells from E9.5, E10.5 and E11.5 can be observed on a dimensional reduction UMAP in FIG. 15. The number of detected genes for each cell per timepoint can be observed in FIG. 16. The coverage for the estimation of the latter was ˜10 k reads per cell. Cell-type annotation based on gene expression markers for each cluster for the E11.5 timepoint can be observed in FIG. 17. The reads mapping to protein coding genes were also homogeneously covering the entire gene body, showing the potential of the method to resolve alternative splicing in single-cells (FIG. 18).

### Example 6: Improvements in Library Preparation

Because the clean-up of the depleted rRNAs (as amplified RNA after in vitro transcription) using the VASA-seq droplet workflow was incomplete due to the large size of the barcodes, and because superVASA has larger barcodes (187 bp), the downstream library preparation procedure had to be optimized to efficiently deplete fragmented rRNA amplified RNA molecules.

To this end, the depletion and ligation steps from the VASA-seq protocol were inverted, and the DNAse digestion step was removed. After the RT was completed and the cDNA retrieved from the droplets, the cDNA was digested by adding 1 μl of exonuclease 1 (NEB) and incubating at 37° C. for 30 minutes. The cDNA is then purified using 1× AMpureXP volumetric ratio and processed using a second-strand synthesis kit and amplified using a HiScribe T7 in vitro transcription kit and incubated overnight at 37° C. After IVT, the purified aRNA's concentration was adjusted to 100 ng/μl and 5 μl of product were mixed with 1 μl of RA3 ligation oligonucleotide (/5rApp/TGGAATTCTCGGGTGCCAAGG/3SpC3/) and the reaction was brought to 70° C. and directly cooled on ice after. 1 μl of 10× T4 RNA ligase reaction buffer (NEB), 1 μl NEB T4 RNA Ligase2, truncated (NEB), 1 μl of RNAseOUT (Invitrogen) and 1 μl of nuclease-free water were supplemented to the reaction and the latter was incubated at 25° C. for 1 hour. The product was then purified with 1.2× volumetric ratio of AmpureXP and eluted in 6 μl of nuclease-free water. The latter was mixed with 4 μl of rRNA depletion probes (12.5 μM), incubated at 95° C. for 2 minutes and brought to 45° C. with a gradient of 0.1° C./s. Once the probes are hybridised 2 μl of Epicentre RNAseH was added to the mix as well as 8 μl of 1.25× RNAseH buffer. The reaction was incubated at 45° C. for 30 minutes and further kept on ice. 2 μl of DNAse (Promega) was further added to the reaction mixture, with 2.2 μl of 10× DNAse buffer (Promega). The mixture was further incubated at 37° C. for 30 minutes. A 1.2× volumetric ratio AmpureXP clean-up was then performed and the aRNA was eluted in 5 μl of nuclease-free water. The adapter ligated aRNA was then mixed with 1 μl of dNTPs (10 mM each, Thermo Fisher Scientific) and 2 μl of RTP oligonucleotide (20 μM, GCCTTGGCACCCGAGAATTCCA). The mixture was then incubated at 65° C. for 5 minutes before being placed directly on ice. 4 μl of 5× First strand synthesis buffer (Invitrogen) were then added to the mix, along with 1 μl of nuclease-free water, 1 μl of 0.1M DTT (Invitrogen), 1 μl of RNAseOUT and 1 μl of Superscript III. The reaction was then heated to 50° C. for 1 hour followed by 70° C. for 15 minutes. 1 μl of RNAseA (ThermoFisher scientific) was further added to each tube and the cDNA was incubated at 37° C. for 30 minutes. The reaction was then purified using a 1× volumetric ratio of AmpureXP beads and eluted in 10 μl. The final product was amplified using a dual-indexed PCR primer pair to minimize index hopping containing the P5 and P7 flow-cell adapters as overhangs.

Results:

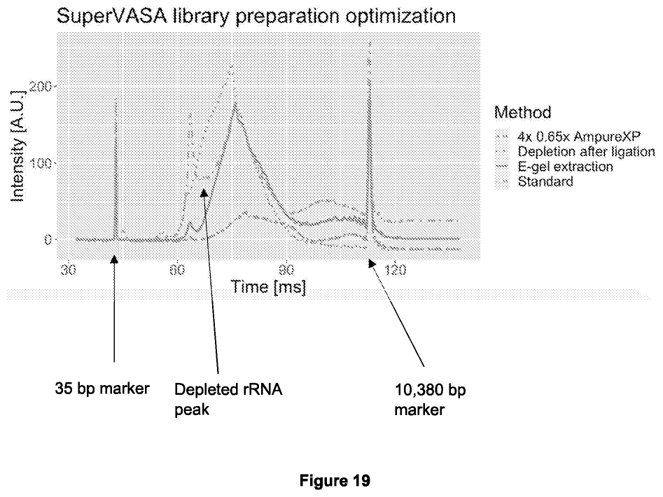

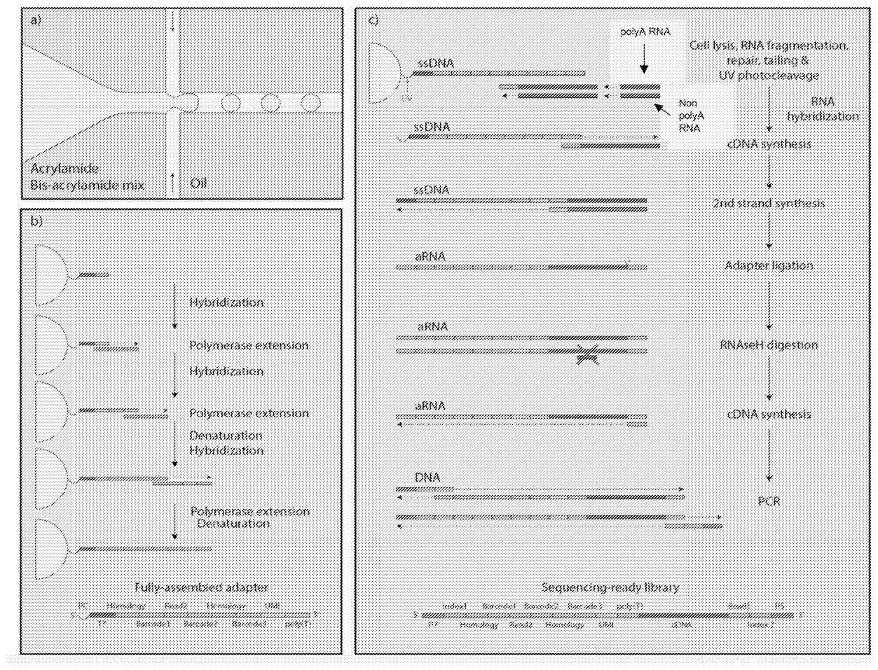

Inverting the rRNA depletion and adapter ligation steps allowed for the effective removal of rRNA peaks, as can be observed from bioanalyzer traces (high-sensitivity kit) in FIG. 18. Although less pronounced with VASA-seq, the large barcode size when triple barcoding is employed prevents the effective removal of depleted aRNA using DNA purification tools. This method, termed “depletion after ligation” in FIG. 19 shows a notable improvement in library size distributions when compared to purification methods such as AmpureXP or E-Gel (1% agarose). An overview of the bead manufacturing and molecular steps is given in FIG. 20. 20.a) is a schematic of the single aqueous inlet used for droplet generation of PAAm droplets. 20.b) shows an illustration of the triple bead barcoding process utilised in the superVASA process. 20.c) proposes an overview of the molecular steps involved in the whole-transcriptome barcoding process, with the RNaseH depletion steps interchanged with the adapter ligation steps.

The project leading to this application has received funding from the European Union's Horizon 2020 research and innovation programme under the Marie Sklodowska-Curie grant agreement No 750772.

The project leading to this application has received funding from the European Research Council (ERC) under the European Union's Horizon 2020 research and innovation programme (grant agreement No 695669).

1. A method of preparing a sequencing library, the method comprising:
a) encapsulating in a microfluidic droplet:
a cell or cell structure comprising RNA; and
lysis and optionally RNA fragmentation reagent;
b) incubating the droplet to release the RNA from the cell or cell structure;
c) optionally fragmenting the RNA in the droplet;
d) adding an RNA tagging reagent into the droplet, wherein the RNA tagging reagent adds an oligonucleotide tag to the RNA;
e) incubating the droplet to allow the RNA to be tagged with the oligonucleotide;
f) hybridizing the oligonucleotide tag of the RNA to a primer adapted to initiate cDNA synthesis (cDNA synthesis primer); and
g) performing reverse transcription to obtain a cDNA sequencing library wherein the cDNA in the cDNA sequencing library comprise a barcode and optionally a UMI.

2. The method of claim 1, wherein:
a) the RNA tagging reagent is an RNA repair and polyadenylation reagent and step e) allows RNA repair and polyadenylation; and
b) the cDNA synthesis primer is a poly-T primer which hybridizes to the poly-A tag of the RNA.

3. The method of claim 2, wherein the poly-T primer further comprises the UMI and/or barcode.

4. The method of claims 1-3, wherein in step d), the RNA tagging reagent is added into the droplet by picoinjection.

5. The method of claims 1-4, wherein step a) additionally comprises encapsulating a bead wherein the bead comprises the cDNA synthesis primer.

6. The method of claim 5 wherein the amount of lysis and optionally fragmentation reagent added into the droplet during encapsulation is:
a) 0.05-0.4 nl; or
b) 20-50% the volume of the droplet.

7. The method of claims 5-6, wherein step d) comprises picoinjecting the following amount of RNA tagging reagent:
a) 0.1-0.5 nl; or
b) 10-50% the volume of the droplet.

8. The method of claims 5-7, wherein reverse transcriptase reagent is added by picoinjection, optionally with the RNA tagging reagent or in a separate picoinjection step after step d).

9. The method of claims 1-4, wherein reverse transcriptase reagent is added by droplet fusion after step d).

10. The method of claim 9, wherein a bead is additionally added by droplet fusion wherein the bead comprises the cDNA synthesis primer.

11. The method of claims 9-10 wherein the amount of lysis and fragmentation reagent added into the droplet during encapsulation is:
a) 5-25 μl; or
b) 20-75% the volume of the droplet.

12. The method of claims 9-11, wherein step d) comprises picoinjecting the following amount of RNA tagging reagent:
a) 5-50 μl; or
b) 10-50% the volume of the droplet.

13. The method of claims 10-12, wherein the frequency of picoinjection is at least 1 kHz, optionally 2 kHz.

14. The method of claim 8 or 9, wherein the following amount of reverse transcriptase reagent is added:
a) 0.5 nl-1.5 nl; or
b) 20-150% the volume of the droplet.

15. The method of claims 1-14, wherein the lysis and optionally fragmentation reagent comprises any one or more of the following:
a) a protease;
b) a divalent metal ion;
c) a non-ionic detergent;
optionally wherein the lysis and optionally fragmentation reagent is added to the droplet to result in any one or more of the following concentrations in the droplet:
a) 0.5-30 U/ml of protease;
b) 0.5-40 mM of divalent metal ion; and/or
c) 0.05-1.5% v/v of non-ionic detergent.

16. The method of claim 15, wherein the protease is Proteinase K; and/or the divalent metal ion is Mg²⁺; and/or the non-ionic detergent is IGEPAL.

17. The method of claims 2-16, wherein the RNA repair and poly(A) polymerase reagent comprises the following:
a) RNA repair enzyme;
b) Polyadenylation enzyme; and
c) ATP;
optionally wherein the RNA repair and poly(A) reagent is added to the droplet to result in any one or more of the following concentrations in the droplet:
a) 0.1-4 kU/ml of repair enzyme;
b) 10-500 U/ml of polyadenylation enzyme; and
c) 0.001-5 mM ATP.

18. The method of any of the preceding claims, wherein step e) comprises incubating the droplet at a temperature of 16-25° C. for 10-60 minutes; followed by 25-39° C. for 5-10 minutes; optionally followed by an ice bath for at least 2 minutes.

19. The method of any of the preceding claims, wherein in step g) the reverse transcriptase reagent added results in a concentration in the droplet of 1-40 kU/ml of reverse transcriptase.

20. The method of any of the preceding claims, wherein the method further comprises:
a) a sorting step (step a)i) downstream of encapsulation step a) wherein the sorting step comprises dividing the droplets into a first droplet set and a second droplet set; or
b) a sorting step (step d)i) downstream of step d), wherein the sorting step comprises dividing the droplets into a first droplet set and a second droplet set; or
c) a sorting step (step g)i) downstream of adding reverse transcriptase step g), wherein the sorting step comprises dividing the droplets into a first droplet set and a second droplet set, optionally wherein in the first droplet set, the droplets comprise lysate from live cells; and
wherein in the second droplet set, the droplets comprise lysate from dead cells, and/or empty droplets and/or cell doublets.

21. The method of any of the preceding claims, wherein a second DNA strand is synthesised by using a reverse transcriptase comprising template switching activity and a template switching oligonucleotide (TSO).

22. A modular microfluidic system for preparing a sequencing library, the modular system comprising:
a) a droplet generation module adapted for encapsulation of cells or cell structures, lysis reagent and optionally beads in microfluidic droplets, the droplet generation module comprising a droplet generation junction in fluid communication with one or more input channels, the one or more input channels for flowing cells, lysis reagent, partitioning fluid and optionally beads into the droplet generation junction
b) a picoinjection module adapted to receive the droplet from the first device, the picoinjection module comprising:
i) a supply channel, into which microfluidic droplets comprising cell lysate and fragmented RNA can be injected wherein the supply channel comprises a droplet spacer; and
ii) a picoinjector for injecting RNA tagging reagent into the droplets wherein the picoinjector is in fluid communication with the supply channel and is downstream of the droplet spacer.

23. The system of claim 22, wherein the end portion of the droplet generation module and/or picoinjection module increases in diameter towards the exit of the device to prevent merging of droplets on collection.

24. The system of claims 22-23, comprising a third microfluidic module, the third microfluidic module comprising:
a) a further picoinjection module comprising:
i) a supply channel into which microfluidic droplets comprising cell lysate and tagged RNA can be injected, the supply channel comprising a droplet spacer; and
ii) a picoinjector, wherein the picoinjector is in fluid communication with the supply channel and is downstream from the droplet spacer, the picoinjector for injecting the reverse transcriptase reagent;
or
b) a droplet fusion module, the droplet fusion module comprising a fusion chamber adapted to fuse reverse transcriptase reagent and a bead with the microfluidic droplet, wherein the droplet fusion module comprises a droplet spacer upstream of the fusion chamber.

25. The system of claims 22-24, wherein the third microfluidic device comprises a dilution oil channel upstream of the droplet spacer.

26. The system of claims 22-25, wherein the distance between the droplet spacer and the picoinjector is about 5-20 times the diameter of the supply channel.

27. The system of claims 22-26, wherein:
a) the distance between the droplet spacer of the picoinjection module and the picoinjector is about 0.8-1 mm; and/or
b) the distance between the droplet spacer of the further picoinjector module of the third microfluidic device and the picoinjector is about 1.8-2 mm.

28. The system of claims 22-27, wherein:
a) the droplet generation module further comprises a bifurcating sorting junction downstream of the droplet generation junction, the bifurcating sorting junction in fluid communication with a first exit channel and a second exit channel wherein the bifurcating sorting junction is adapted to divide the droplets into a first droplet set which exits via the first exit channel and a second droplet set which exits via the second exit channel; or
b) the picoinjection module comprises a bifurcated sorting junction downstream of the picoinjector, the bifurcating sorting junction in fluid communication with a first exit channel and a second exit channel wherein the bifurcating sorting junction is adapted to divide the droplets into a first droplet set which exits via the first exit channel and a second droplet set which exits via the second exit channel; or
c) the third microfluidic module comprises a bifurcating sorting junction downstream of the picoinjector or fusion junction, the bifurcating sorting junction in fluid communication with a first exit channel and a second exit channel wherein the bifurcating sorting junction is adapted to divide the droplets into a first droplet set which exits via the first exit channel and a second droplet set which exits via the second exit channel.

29. The system of claim 28a), wherein the first channel further comprises a droplet channel, in fluid communication with the first exit channel and adapted to add empty droplets to the droplets to be analysed to bulk out the sample.

30. The system of claims 22-29, wherein the system further comprises a droplet collection and re-injection device, the device comprising the device comprising a container for holding an immiscible liquid with lower density than water, the container comprising a tip, the tip connectable to the exit of the first microfluidic device and the injection port of the droplet generation or picoinjection microfluidic modules, the container connectable to a pump, the pump adapted to eject droplets from the tip during injection into the subsequent microfluidic device, optionally wherein the pump is additionally adapted to aspirate droplets into the droplet collection device during collection.

31. The method of any of claims 1-21, implemented with the system of claims 22-30.

32. The method of any of claims 1-22, implemented with the system of claims 22-23, 28a or 29 wherein the reverse transcriptase is added to the droplet by picoinjection with the RNA tagging reagent in the second microfluidic device.

33. A method of preparing a sequencing library, the method comprising:
a) encapsulating in a microfluidic droplet:
i) a cell or cell structure comprising RNA; and
ii) lysis and RNA tagging reagents, wherein the RNA tagging reagent adds an oligonucleotide tag to the RNA;
b) incubating the droplet to release the RNA from the cell or cell structure and to allow the RNA to be tagged with the oligonucleotide;
c) hybridizing the oligonucleotide tag of the RNA to a cDNA synthesis primer; and
d) performing reverse transcription to obtain a cDNA sequencing library
wherein each cDNA in the cDNA sequencing library comprises a barcode and optionally a UMI.

34. The method of claim 33, wherein reverse transcriptase reagent is added into the droplet by picoinjection or droplet fusion after step b).

35. The method of claim 34, implemented with the device of claims 22-23, 28a, 29 or 30.

36. The method of any of claim 31, 32 or 35, wherein:
a) the microdroplets are collected from the droplet generation module or picoinjection module with a droplet collection device, the droplet collection device comprising a container, the container comprising an immiscible liquid with lower density than water, optionally a hydrocarbon or silicone oil, the container comprising a tip, wherein the tip is connected to the exit of the droplet generation module to collect droplets into the device;
b) the microfluidic droplets are incubated to allow lysis and fragmentation; or repair and polyadenylation respectively; and
c) optionally the droplets are reinjected into the picoinjection device or third microfluidic module by connecting the container to a pump adapted to eject droplets from the tip.
